(19) United States
(12) Patent Application Publication
(10) Pub. No.: US 2010/0065428 A1
(43) Pub. Date: Mar. 18, 2010

(54) ISOELECTRIC FOCUSING GELS AND METHODS OF USE THEREOF

(75) Inventors: Tom DILLER, San Diego, CA (US); Tom BEARDSLEE, Carlsbad, CA (US); Regina D. ROONEY, La Jolla, CA (US); Joseph W. AMSHEY, Encinitas, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/512,797

(22) Filed: Jul. 30, 2009

Related U.S. Application Data
(63) Continuation of application No. 10/961,308, filed on Oct. 7, 2004.
(60) Provisional application No. 60/510,674, filed on Oct. 9, 2003.
(60) Provisional application No. 60/509,512, filed on Oct. 7, 2003.

(51) Int. Cl. C07K 1/28

(52) U.S. Cl. 204/459

(57) ABSTRACT

Provided herein are rapidly-rehydratable prior-cast, dehydrated, electrophoresis separation media particularly useful for isoelectric focusing, including methods of making, and methods of use thereof.

### CROSS-REFERENCE TO RELATED APPLICATIONS

This application claims the benefit of U.S. provisional patent application Ser. No. 60/509,512, filed Oct. 7, 2003 and Ser. No. 60/510,674, filed Oct. 9, 2003, the disclosures of which are incorporated herein by reference in their entireties.

### FIELD OF THE INVENTION

The present invention is in the field of electrophoresis, and relates particularly to rapidly-rehydratable prior-cast electrophoresis separation media.

### BACKGROUND OF THE INVENTION

Separation of proteins by two-dimensional gel electrophoresis (“2DE” or “2D-PAGE”) coupled with subsequent mass spectrometric determination of the identity of the separated proteins is central to proteomics research. In 2DE, a protein mixture is separated in the first dimension by the intrinsic charge characteristics of the proteins by a process known as isoelectric focusing (“IEF”). After the focusing step, the gel strip containing the separated proteins is transferred to a slab gel through which the proteins are further separated in the second dimension on the basis of their molecular mass.

Separation of proteins by IEF in the first dimension is usually performed using thin strips of polyacrylamide gel that contain an immobilized pH gradient (“IPG gel strips”). See, e.g., U.S. Pat. No. 4,130,470, U.S. Pat. No. 5,785,832, PCT International Publication No. WO89/09206, Righetti et al. (1984) J. Chromatogr. 291:31-42. Such strips are available commercially in a dehydrated state, and must be rehydrated prior to use. Protein samples may either be applied after this rehydration step, or they may be included in the solution used for rehydration. Sanchez et al. (1997) Electrophoresis 18:324-27.

With conventional strips, problems with resolution, such as vertical streaking in the second dimension, may result from protein overloading. It has been suggested that these problems may be solved through the use of narrow-range immobilized pH gradients with large sample loading volumes, Bjellqvist et al. (1993) Electrophoresis 14:1375-78, but these solutions may frequently prove impractical. Certain changes in the gel formulation may address some problems with resolution, Esteve-Romero et al. (1996) Electrophoresis 17:704-8, but other problems remain.

With conventional IPG strips, the rehydration step can be the slowest part of the entire process, requiring at least 11 hours or even longer.

Although central to proteomic analysis, the method of 2DE has traditionally been time-consuming, cumbersome, expensive, irreproducible, and operator-dependent, in part because of the long times required for rehydration of the IPG gel strips.

There is thus a need in the art for apparatus, compositions, and methods that shorten the time required to rehydrate an IPG gel strip and the overall time required to perform 2-dimensional electrophoresis. There is a further need in the art for apparatus, compositions, and methods for improving the resolution of proteins in IEF on IPG gel strips.

### SUMMARY OF THE INVENTION

The present invention solves these and other needs in the art by providing novel gel matrix formulations that permit surprisingly rapid rehydration of prior-cast, dehydrated gels, particularly of prior-cast, dehydrated immobilized pH gradient (IPG) gels fashioned as IPG strips. Immobilized pH gradient gels and strips produced using these formulations additionally display precise and accurate pH ranges and high resolution IEF of proteins with minimal streaking.

Accordingly, in a first aspect, the invention provides a gel suitable for isoelectric focusing, comprising a polymerized acrylamide matrix cast from an acidic solution and a basic solution. The acrylamide matrix typically ranges from not less than about pH 3.5 to not more than about pH 7.5 upon rehydration, or the acrylamide matrix has a pH range that spans at least 5, 6, or 7 pH units, for example the matrix can have a pH from about 3.0 to about 10.0. The basic solution, in one example, comprises a plurality of acrylamido buffers with a combined concentration of at least about 32 mM. In another example, the basic solution, the acidic solution, or both the basic solution and the acid solution have a beta value of greater than 3, greater than 4, or greater than 5 mEq/L/pH. For example, the basic solution, the acidic solution, or both the basic solution and the acidic solution, or the final poured gel that includes both the basic solution and the acidic solution has a beta value of 4, 5, 6, or 7 mEq/L/pH. In illustrative embodiments, the basic solution, the acidic solution, or the combination of both the basic solution and the acidic solution have a beta value of 5 mEq/L/pH.

In some embodiments, the basic solution comprises at least three acrylamido buffers, and in some embodiments the acrylamido buffers have a combined concentration of at least about 35 mM, or in some examples, at least about 40 mM. In some examples, the acrylamido buffers have a beta value of greater than 3, 4, or 5 mEq/L/pH, for example 5 mEq/L/pH.

In some embodiments of the invention, the acrylamide matrix of the gel ranges in pH from not less than about pH 3.5 to not more than about pH 7.5. In preferred embodiments, the pH ranges from not less than about pH 3.0 to not more than about pH 10.0. In still other preferred embodiments, the pH ranges from not less than about pH 6.0 to not more than about pH 10.0, from not less than about pH 4.0 to not more than about pH 7.0, from not less than about pH 4.5 to not more than about pH 5.5, from not less than about pH 5.3 to not more than about pH 6.3, or even from not less than about pH 6.1 to not more than about pH 7.1.

In one series of embodiments, the gel has been dehydrated after casting, so as to have little or no water.

Such dehydrated gels are, in some embodiments, capable of rehydrating after contact with aqueous buffer in no more than 8 hours at room temperature, with other embodiments capable of rehydrating after contact with aqueous buffer in no more than 2 hours at room temperature, and others after no more than 60 minutes at room temperature.

In some embodiments, the gel is attached to a support, such as a plastic film. The gel and support may be fashioned as a strip.

In another aspect, the invention provides a hydratable gel strip, comprising a dehydrated acrylamide matrix attached to a support, wherein the gel strip is capable of rehydrating in no more than 8 hours at room temperature. In some embodiments, the gel strip is capable of rehydrating in no more than 2 hours at room temperature, or even in no more than 1 hour at room temperature.

In some embodiments, the acrylamide matrix ranges from not less than about pH 3.5 to not more than about pH 7.5 upon rehydration.

In typical embodiments, the hydratable gel strip is cast from a basic solution and an acidic solution, the basic solution comprising at least three acrylamido buffers with a combined concentration of at least about 32 mM.

In another aspect, the present invention provides a method for performing gel electrophoresis of a sample, wherein the method includes rehydrating a dried gel strip; and separating one or more proteins in the sample within the rehydrated gel strip based on their isoelectric point, wherein the method is completed in no more than 6, 5, 4, 3, or 2 hours. In illustrative examples, the method is completed in about 3 hours. In certain examples, the method further includes placing the rehydrated gel comprising the separated one or more proteins on a slab gel, and electrophoresing the one more proteins into the slab gel, wherein the method is completed in no more than 10, 9, 8, 7, 6, 5, or 4 hours. In illustrative embodiments, the method is completed in about 4 hours.

In another aspect, the invention provides a method of making a dehydrated gel strip, the method comprising, casting an acrylamide matrix from an acidic solution and a basic solution onto a support, wherein the acrylamide matrix comprises a plurality of acrylamido buffers with a combined concentration of at least about 32 mM; and then drying the gel matrix.

The basic solution may comprise at least three acrylamido buffers, and the combined concentration of the acrylamido buffers in the basic solution may be at least about 35 mM, at least about 40 mM, or more. In some embodiments, the acrylamide matrix may range in pH from not less than about pH 3.5 to not more than about pH 7.5.

In certain embodiments, the support is a plastic film, such as a plastic film that comprises vinyl moieties capable of copolymerization into the gel matrix. In a variety of embodiments, the matrix and support are fashioned as a strip.

In yet a further aspect, the invention provides a gel having a pH that varies progressively along the length of said gel.

The gel comprises a polymer matrix cast from an acidic solution and a basic solution, wherein the polymer matrix comprises at least one polyacrylamide species and the basic solution comprises a plurality of acrylamido buffers with a combined concentration of at least about 32 mM.

The gel may have a linear pH range or non-linear pH range, the pH ranging in some embodiments from not less than about pH 3.5 to not more than about pH 7.5.

In some embodiments, the gel comprises little or no liquid, and is capable of rehydrating in not more than about 8 hours at room temperature, often in not more than about 6 hours at room temperature, alternatively in not more than 2 hours at room temperature, or even in no more than about 60 minutes at room temperature.

Gels of this aspect of the invention may be attached to a support.

In a further aspect, the invention provides a gel having a pH that varies progressively along the length of said gel. The gel comprises a polymer matrix cast from an acidic solution and a basic solution, the polymer matrix comprising at least one polyacrylamide species. The gel, once dried, is capable of rehydrating in no more than about 8 hours at room temperature, often in no more than about 2 hours at room temperature, or even in no more than about 60 minutes at room temperature.

The gel may have a linear or non-linear pH range, and may have a pH that ranges from not less than about pH 3.5 to not more than about pH 7.5. In other embodiments, the gel may have a pH that ranges from not less than about 3.0 to not more than about 10.0, from not less than about 6.0 to not more than about 10.0, from not less than about 4.0 to not more than about 7.0, from not less than about 4.5 to not more than about 5.5, from not less than about 5.3 to not more than about 6.3, or even from not less than about 6.1 to not more than about 7.1.

In another aspect, the invention provides a method of making a gel having a pH that varies progressively along the length of said gel, the method comprising, casting a polymer matrix from a varying mix of an acidic solution and a basic solution, wherein the polymer matrix comprises at least one polyacrylamide species and the basic solution comprises a plurality of acrylamido buffers with a combined concentration of at least about 32 mM. The gel may have a linear pH range or non-linear pH range, which may range from not less than about pH 3.5 to not more than about pH 7.5.

The method may further comprise drying the gel in order to produce a dry gel that comprises little or no liquid. The dry gel in some embodiments is capable of rehydrating in no more than about 8 hours at room temperature, in other embodiments in no more than about 2 hours at room temperature, and in yet other embodiments in no more than about 60 minutes at room temperature.

The gel may be attached to a support and optionally fashioned as a strip.

In a still further aspect, the invention provides a method of preparing a gel for use in electrophoresis, the method comprising, rehydrating the dehydrated gel of the invention. The dehydrated gel may be attached to a support. The rehydration solution may optionally comprise at least one analyte.

In another aspect, the invention provides a method of separating two or more molecules from each other. The method comprises electrophoresing a sample comprising said two or more molecules through the gel and strips of the present invention. Said sample may usefully be a biological sample, including a sample comprising proteins.

The method may further comprise a subsequent step of electrophoresing the sample in a second dimension, for example a second dimension that separates according to size.

The method may further comprise transferring the analytes to a membrane.

In some embodiments, the method may comprise contacting the gel with a compound that binds to proteins, including compounds that are detectable. In some embodiments, the compound may specifically bind to one or more proteins in a protein sample electrophoresed in the gel.

In another embodiment, provided herein is a kit that includes one or more dried gel strips of the invention. In certain examples, the kit includes 2, 3, 4, 5, 6, 7, 8, 9, 10, 11, 12, 13, 14, 15, 16, 17, 18, 19, or 20 gel strips. The strip can be labeled with a unique identifying number, pH range, and/or orientation marks. The gel strips can be supplied attached to a tri-fold card to help facilitate access and removal.

### BRIEF DESCRIPTION OF THE DRAWINGS

These and other objects and advantages of the present invention will be apparent upon consideration of the following detailed description, taken in conjunction with the accompanying drawings:

FIG. 1. Expansion of the pH gradient with narrow pH range IPG strips;

FIG. 2. Rat liver lysate separated on 2D gels. Top gel: whole lysated focused in the first dimension on a 4-7 IPG strips. Bottom gels: whole lysate pre-fractionated by solution-phase IEF with the ZOOM® IEF Fractionator followed by separation on 2D gels using the appropriate narrow pH range IPG strips.

FIG. 3. Overlay grid measurement of protein position on 2D gels from 1-pH unit narrow-range strips. The solid is the expected protein position.

FIG. 4. Load capacity of narrow range IPG strips with purified proteins.

FIG. 5. Protein overload. Each IPG strip (pH 4-7 on left, pH 5.3-6.3 on right) was rehydrated with 7.75 mg of bovine serum albumin.

FIG. 6. IEF Separation of protein standards on narrow pH range IPG strips with various focusing times.

FIG. 7. Pre-fractionated rat liver lysate separated on narrow pH range IPG strips. ZOOM® IEF Fractionator samples with 0.2% ZOOM® Carrier Ampholytes 3-10+1.0% ZOOM® Carrier Ampholytes 4-7 spiked into the pre-fractionated samples prior to rehydration A. 4.6-5.4 fraction, focused on 4.5-5.5 B. Microsol 5.4-6.2 fraction, focused on 5.3-6.3 strips strips. C. Microsol 6.2-7.0 fraction, focused on 6.1-7.1 strips.

FIG. 8. Comparison 2D gel separation of rat liver lysate applied directly to pH a 4-7 IPG strips or pre-fractionated by solution-phase IEF and applied to pH 4.5-5.5 IPG Strip. Upper panel: 48 μg rat liver lysate in 7 M Urea, 2M thiourea, 4% CHAPS, 1% ZOOM® Carrier Ampholytes 3-10. Lower panel: The 4.6 to 5.4 fraction of rat liver lysate pre-fractionated in the ZOOM® IEF Fractionator was removed and ZOOM® Carrier Ampholytes 3-10 (0.2%), ZOOM® Carrier Ampholytes 4-7 (1.0%) and bromphenol blue (trace) were added. The second dimension for both gels was 4-12% Bis-Tris NuPAGE stained with SimplyBlue.

FIG. 9. E. coli lysate (30 μg) focused on 4.5-5.5 (left) and 5.3-6.3 (right) strips. Ampholytes used in the rehydration solution are indicated on the individual gels.

FIG. 10. E. coli lysate (30 μg) focused on 6.1-7.1 strips. Ampholytes used in the rehydration solution are as indicated on the individual gels.

FIG. 11. Load capacity with crude lysates. Strips were rehydrated with 300 μg of E. coli lysate.

FIG. 12. A comparison of 2D gels run with expanded pH gradient using 1-pH unit IPG strips and a 2D gel run with pH 4-7 IPG strips. The 4.5-5.5 and 5.3-6.3 strips were rehydrated with 30 μg of E. coli lysate, and the 6.1-7.1 strip was rehydrated with 100 μg of the same lysate.

FIG. 13. Placement of windows and wicks in the IPGRunner™. The schematic depicts the placement of the window in the cassette cover with respect to the IPG strip (Top view) and the wick placement over the extreme ends of the gel (Both views).

FIG. 14. Rehydration time course by 2D gels of pH 4-7 IPG strips.

FIG. 15. Spot counting of 2D gel sections for a rehydration time course using pH 4-7 IPG strips (7 cm).

FIG. 16. Mass spectrometric analysis of a rehydration time course using pH 4-7 IPG strips (7 cm) loaded with 75 μg of E. coli lysate.

FIG. 17. Rehydration comparison of pH 5.3-6.3 IPG strips.

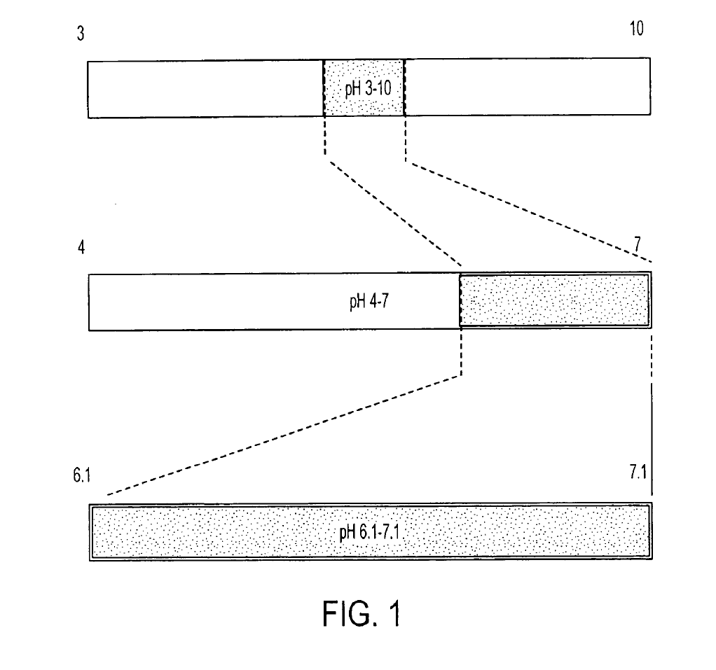

### DETAILED DESCRIPTION

The current invention provides novel compositions for the polymerized acrylamide matrix of isoelectric focusing gels, including immobilized pH gradient (IPG) gel strips. Gels and strips produced using these formulations display precise and accurate pH ranges, provide high resolution IEF of proteins with minimal streaking, and, surprisingly, allow for the rapid rehydration of strips after dehydration, substantially reducing the time required to perform first dimension separations in 2DE applications.

In one series of embodiments, the polymerized acrylamide matrix is cast upon a support, often a flexible support, typically a plastic support; the plastic support may, for example, be a polyester film or a polyester mesh fabric. In embodiments particularly designed for use with existing electrophoresis equipment, the gel and support of such embodiments are fashioned as a strip, that is, with a first dimension substantially greater than a second dimension. However, the dimensions of gel and backing are not critical to the invention, which may be practiced with gels of any dimension. For ease of reference, all support-backed gels of the present invention will be referred to herein as “strips” or “IPG strips” without implying a particular size or dimension.

Although the IPG strips of the present invention may be of any dimension, in some embodiments the strips have approximate lengths of, for example, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 180 mm, 200 mm, 220 mm, 240 mm, or even longer. In some embodiments of the invention, the strips have approximate widths of, for example, 1.0 mm, 1.5 mm, 2 mm, 2.5 mm, 3 mm, 3.5 mm, 4 mm, 4.5 mm, 5 mm, or even wider and will have approximate thicknesses of 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm or even thicker.

In typical embodiments, further described below, strips of the present invention are provided in dehydrated form, to be rehydrated prior to electrophoretic focusing of analytes therein. Neither complete removal of moisture, during dehydration, nor complete saturation with liquid, during rehydration, is required or intended.

### IPG Gel Compositions

In a first aspect, the invention provides a gel suitable for isoelectric focusing, the gel comprising a polymerized acrylamide matrix. The acrylamide matrix typically ranges from not less than about pH 3.5 to not more than about pH 7.5 upon rehydration, or the acrylamide matrix has a pH range that spans at least 5, 6, or 7 pH units, for example the matrix can have a pH from about 3.0 to about 10.0. In typical embodiments, the polymerized acrylamide matrix ranges in pH from not less than about pH 3.5 to not more than about pH 7.5, and is cast from an acidic solution and a basic solution. Generally, each of the acidic and basic solutions comprises at least one acrylamido buffer; in typical embodiments, the basic solution comprises two or more acrylamido buffers with a combined concentration of at least about 32 mM.

The polymerized acrylamide matrix of the gels and strips, of the present invention is cast from an acidic solution and a basic solution, each of the solutions comprising at least one acrylamido buffer.

When comparing any two solutions used to cast the polymerized acrylamide matrix of a gel or strip of the present invention, the “acidic solution” is the solution that contains a relatively higher combined concentration of acidic buffers, and the “basic solution” is the solution that contains a relatively higher combined concentration of basic buffers. As should be clear from the above definition, an “acidic solution” does not necessarily display an acid pH value. Likewise, a “basic solution” does not necessarily display a basic pH value. Furthermore, an “acidic solution” can contain basic buffers, and a “basic solution” can contain acidic buffers.

Exemplary acrylamido buffers used in the acidic and basic solutions used to cast the isoelectric focusing gels and IPG gel strips of the present invention are listed in Table 1. These buffers belong to a set of non-amphoteric weak acids and bases having a vinyl moiety for incorporation into the gel matrix and are available commercially (Amersham Biosciences, Piscataway, N.J., USA and Sigma-Aldrich, St. Louis, Mo., USA). See also, Chiari et al. (1989) Applied and Theoretical Electrophoresis 1, 99-102 and Chiari et al. (1989) Applied and Theoretical Electrophoresis 1, 103-107.

TABLE 1
Acrylamido buffers

| | Chemical name | pKₐ | CAS# |
|---|---|---|---|
| Acidic | 2-Acrylamido-2-methylpropane | 1.0 | 15214- |
| Buffers | sulfonic acid | | 89-8 |
| | 2-Acrylamido glycolic acid | 3.1 | 6737- |
| | | | 24-2 |
| | Glycine-N-acryloyl | 3.6 | 24599- |
| | | | 25-5 |
| | β-Alanine-N-acryloyl | 4.4 | 16753- |
| | | | 07-4 |
| | 4-Acrylamido-n-butyric acid | 4.6 | 53370- |
| | | | 87-9 |
| Basic | 2-Morpholino ethylacrylamide | 6.2 | 13276- |
| Buffers | | | 17-0 |
| | 3-Morpholino propylacrylamide | 7.0 | 46348- |
| | | | 76-9 |
| | N,N-Dimethyl aminoethyl | 8.5 | 925-76-8 |
| | acrylamide | | |
| | N,N-Dimethyl aminopropyl | 9.3 | 3845- |
| | acrylamide | | 76-9 |
| | N,N-Diethyl aminopropyl | 10.3 | 7065- |
| | acrylamide | | 11-4 |
| | N-[2-Diethylaminoethyl]acrylamide | 12.0 | 10595- |
| | | | 45-6 |

Other known acrylamido buffers, not shown in the table but having known pKₐ values, may also be used in casting the acrylamide matrix according to methods known in the art—(see, e.g., Righetti (1990) Immobilized pH gradients. Theory and methodology, Elsevier, Amsterdam, New York, Oxford, pp. 53-116) as may acrylamido buffers yet to be synthesized. A desired molarity for the buffer and/or buffer capacity (i.e., beta value (See e.g., Rhigetti (1990), p. 74)) can be identified using the specific molarities and charge densities values provided herein. For example, in certain illustrative embodiments, regardless of the specific buffer used, typically an acrylamido buffer, the acidic and/or basic buffer has a beta value of greater than 3, for example 4, 5, 6, 7, or 8 mEq/L/pH, and/or a molarity of at least 32 mM, 33 mM, 34 mM, 35 mM, 40 mM, 55 mM, 60 mM, 65 mM, 70 mM, or 75 mM.

The acid and basic solutions are combined to cast gels and strips having various pH ranges. In some embodiments, the pH range may have a lower value of about 0, 1, 2, 3, 4, 5, 6, 7, 8, 9, 10, 11, 12, or even 13, with intermediate values permissible. In some embodiments, the pH range may have an upper value of about 1, 2, 3, 4, 5, 6, 7, 8, 9, 10, 11, 12, 13, or even 14, with intermediate values permissible. In a highly preferred embodiment, the pH ranges from not less than about pH 3.5 to not more than about pH 7.5, including, for example, a range of not less than about pH 5 to not more than about pH 6, and including, for example a range of about pH 4.5-5.5 and a range of about pH 5.3-6.3. In other highly preferred embodiments of the invention, the pH ranges are about 3.0-10.0, about 6.0-10.0, about 4.0-7.0, about 6.1-7.1, and about 9.0-12.0.

In some embodiments, the gel has a linear pH range. In other embodiments, the gel has a nonlinear pH range.

In certain preferred embodiments, the acidic and basic solutions used to cast gels and strips of the present invention may contain, but are not limited to, the concentrations of acrylamido buffers shown in Tables 2A and 2B, wherein the listed pKₐ values represent the corresponding acrylamido buffers listed in Table 1.

TABLE 2A
Representative Acrylamido Buffer Compositions of Acidic
and Basic Solutions Used to Cast Gels

| | | Acidic | Basic |
|---|---|---|---|
| | | Solution | Solution |
| | | Conc. | Conc. |
| Type of IPG gel strip | pKₐ | (mM) | (mM) |
| pH 4-7 Gradient (buffer | 3.6 | 13.41 | 7.01 |
| capacity (beta value = 5.42) | 4.6 | 2.55 | 17.12 |
| | 6.2 | 10.44 | 3.50 |
| | 7.0 | 0.00 | 6.24 |
| | 8.5 | 0.00 | 0.00 |
| | 9.3 | 0.00 | 20.32 |
| pH 4.5-5.5 (buffer capacity | 3.6 | 12.18 | 10.08 |
| (beta value = 5.113) | 4.6 | 5.49 | 10.27 |
| | 6.2 | 9.09 | 6.81 |
| | 7.0 | 1.25 | 3.30 |
| | 8.5 | 0.00 | 0.00 |
| | 9.3 | 4.08 | 10.76 |
| pH 5.3-6.3 (buffer capacity | 3.6 | 10.71 | 8.61 |
| (beta value = 5.068) | 4.6 | 8.84 | 13.62 |
| | 6.2 | 7.50 | 5.22 |
| | 7.0 | 2.69 | 4.74 |
| | 8.5 | 0.00 | 0.00 |
| | 9.3 | 8.75 | 15.43 |
| pH 6.1-7.1 (buffer capacity | 3.6 | 8.966 | 15.873 |
| (beta value = 5.187)) | 6.2 | 5.932 | 0.996 |
| | 7.0 | 5.977 | 8.893 |
| | 9.3 | 0.351 | 11.875 |

TABLE 2B
Solutions Used to Cast pH 3-10 Linear Gradient Strips

| | Acidic | Basic |
|---|---|---|
| | Solution | Solution |
| pKₐ | Conc. (mM) | Conc. (mM) |
| 3.6 | 5.234 | 0.0 |
| 4.6 | 17.969 | 5.0 |
| 6.2 | 0.469 | 20.0 |
| 7.0 | 0.0 | 10.0 |
| 8.5 | 0.0 | 7.0 |
| 9.3 | 0.0 | 1.0 |
| 10.3 | 0.0 | 7.0 |

The pH ranges may be altered from these exemplary embodiments either by trial and error or by use of a computer simulation program to calculate concentrations of the acrylamido buffers. See, e.g., Altland (1990) Electrophoresis 11, 140-147; Tonani et al., (1991) Electrophoresis 12, 1011-1021; Giaffreda et al. (1993) J. Chromatogr. 630, 313-327; Righetti et al. (1994) Electrophoresis 15, 1040-1043. Use of alternative acrylamido buffers with different pKₐ values may require further testing and formulation to achieve desired results within a desired pH range, which testing and formulation are routine in the art.

As used herein, the combined concentration of acrylamido buffers in the acidic solution or basic solution represents the sum of the individual concentrations of acrylamido buffers in the respective solution. For example, the combined concentration of acrylamido buffers in the basic solution set forth for preparation of the exemplary pH 4-7 range strips in Table 2A is 54.19 mM.

In typical embodiments of the gels and strips of the present invention, the basic solution comprises at least two acrylamido buffers, but may in some embodiments comprise at least three acrylamido buffers, in some embodiments at least four acrylamido buffers, and in yet other embodiments at least five acrylamido buffers, with a combined concentration of at least about 32 mM, at times at least about 35 mM, 40 mM, even at least about 50 mM or more, with intermediate values possible, including at least about 33 mM, 34 mM, 36 mM, 37 mM, 38 mM, 39 mM, 41 mM, 42 mM, 43 mM, 44 mM, 45 mM, 46 mM, 47 mM, 48 mM, and 49 mM. In certain aspects, the acidic and/or basic acrylamido buffer has a beta value of greater than 3, for example 4, 5, 6, 7, or 8 mEq/L/pH, and/or a molarity of at least 32 mM, 33 mM, 34 mM, 35 mM, 40 mM, 55 mM, 60 mM, 65 mM, 70 mM, or 75 mM. In certain aspects the combination of the acid acrylamido buffer and the basic acrylamido buffer in a poured gel has a beta value of greater than 3, for example 4, 5, 6, 7, or 8 mEq/L/pH. For example the beta value can be between 4 and 8, or between 4 and 7, or in yet another example, between 4 and 6 mEq/L/pH.

In another example, a poured gel includes acrylamido buffers with a buffer capacity, molarity, and/or charge density that is greater than, for example at least 4/3, 5/3, 2 times, 3 times, or 4 times, the buffer capacity, molarity, and/or charge density that is traditionally (i.e. typically or standardly) used for isoelectric focusing gels. Traditional isoelectric focusing gel compositions are defined in, e.g., Righetti (1990) Immobilized pH gradients. Theory and methodology, Elsevier, Amsterdam, New York, Oxford, pp. 53-116, incorporated herein by reference in its entirety). In illustrative aspects, the buffer capacity, molarity, and/or charge density is between 4/3 and 2 times, more particularly, for example 5/3 that typically used for isoelectric focusing. In other examples, the buffer capacity, molarity, and/or charge density is between 33% and 100%, for example 66%, greater than a traditional isoelectric focusing gel.

In another aspect, the gels and gel strips of the present invention have a higher ionic strength than has been previously used. It has been found that this higher ionic strength can be provided by an increased buffering capacity of acrylamido buffers for a given pH. When increased ionic strength of the gels and gel strips is provided by acrylamido buffers, the gel and gel strips retain their isoelectric focusing properties and utility within a 2-D gel apparatus. Methods for calculating ionic strength within an isoelectric focusing gel mixture are known and values may be summed for a given buffer mixture (see, e.g., Righetti (1990) Immobilized pH gradients. Theory and methodology, Elsevier, Amsterdam, New York, Oxford, pp. 53-116, 98-101; and Righetti and Giafferda, “Immobilized buffers for isoelectric focusing: From gradients to membranes,” Electrophoresis 15, 1040-1043 (1994)).

The buffers are combined, according to their pKₐ values, to create a fixed pH gradient by co-polymerization with monoolefinic monomers, such as acrylamide, and di- or polyolefinic monomer crosslinkers, such as methylenebisacrylamide.

Monoolefinic monomers useful in the gel matrices of the present invention include acrylamide, methacrylamide and derivatives thereof such as alkyl-, or hydroxyalkyl derivates, e.g. N,N-dimethylacrylamide, N-hydroxypropylacrylamide, N-hydroxymethylacrylamide. The di- or polyolefinic monomer is preferably a compound containing two or more acryl or methacryl groups such as e.g. methylenebisacrylamide, N,N′-diallyltartardiamide, N,N′-1,2-dihydroxyethylene-bisacrylamide, N,N-bisacrylyl cystamine, trisacryloyl-hexahydrotriazine.

The monoolefinic monomer of gel matrices of the present invention may generally be selected from acrylic- and methacrylic acid derivatives, for example alkyl esters such as ethyl acrylate and hydroxyalkyl esters such as 2-hydroxyethyl methacrylate.

In yet further embodiments, acrylamide monomers may be copolymerized with polysaccharide substituted to contain vinyl groups, for example allyl glycidyl dextran as described in EP 87995, the disclosure of which is incorporated herein by reference in its entirety.

The w/v percentage of the total monomer (% T), such as acrylamide, in the gel matrix can be at least about 3.0%, more typically at least about 4.0%, or even at least about 5.0% or 6.0%, with the % T typically no more than about 6.0%, with intermediate values permissible. The percent w/w of crosslinker to total acrylamide (% C) may be as low as about 2.0%, typically greater than about 2.0%, with % C typically at least about 3.0%, and typically no more than about 4.0%, with intermediate values permissible.

For example, gels cast with a pH range of pH 4-7 using the exemplary formulations of Table 2A contain 4.0% T and 3.0% C, while the narrow-range pH 4.5-5.5 and pH 5.3-6.3 gels contain 5.0% T and 3.0% C.

During preparation of the gel matrix, polymerization initiator agents and/or catalysts (collectively, “polymerization agents”) are typically added to the gel solutions, either directly or during a mixing step as a gradient is formed between the acidic and basic solutions. The polymerization agents typically used to polymerize acrylamide are N,N,N′,N′-tetramethylethylenediamine (“TEMED”) as catalyst and ammonium persulfate (“APS”) as initiator, although other agents having similar activities may be used within the scope of the invention.

In some embodiments, photoinitiators of polymerization are used.

Suitable photoinitiators are known in the art and include, by way of non-limiting example, the following:

Benzoin ethers, benzophenone derivatives and amines, phenantrenequinones and amines, naphtoquinones and amines, methylene blue and toluene sulfinate (EP 0 169 397; the use of the latter two compounds for photopolymerization of polyacrylamide gels is also described by Lyumbimova et al., Electrophoresis 14:40-50, 1993);

DMPAP (2,2-dimethoxy-2-phenyl-acetophenone) and related compounds as disclosed in U.S. Pat. Nos. 3,715,293 and 3,801,329, both to Sandner et al. These patents disclose acetophenones di or tri-substituted at the 2 position, as improvements over acetophenones substituted at the 3, 4 and/or 4,4′ position, analogous xanthophenones, and benzoin and its lower alkyl derivatives;

Phenones, including certain acetophenones, xanthones, fluoroenones, and anthroquinones, in combination with certain amines, for example triethanolamine, are used for rapid photopolymerization of unsaturated compounds, including acrylamide, as described in U.S. Pat. No. 3,759,807 to Osborn and Tercker;

Benzophenones with benzoylcyclohexanol, as described in U.S. Pat. No. 4,609,612 to Berner et al.; Carboxylated analogs of “Mitchler's ketone”, a diaminobenzophenone, which are watersoluble photoinitiators and are described in U.S. Pat. No. 4,576,975 to Reilly; and

Photoinitiators described in U.S. Pat. Nos. 5,916,427 and 6,197,173, both to Kirkpatrick.

In preferred embodiments, the photoinitiator is selected from the group consisting of:

1-hydroxy-cyclohexyl-phenyl-ketone (1-HCPK), a.k.a. 1-hydroxycyclohexyl)phenyl-methanone, CAS Reg. No. 947-19-3 [commercially available as IRGACURE® 184 from Ciba-Geigy (Basel, Switzerland) and as SarCure SR1122 from Sartomer (Exton, Pa.)];

2,2-dimethoxy-2-phenylacetophenone (commercially available as IRGACURE® 651 from Ciba-Geigy);

2-methyl-1-[4-(methylthio)phenyl]-2-(4-morpholinyl)-1-propanone (commercially available as IRGACURE® 907 from Ciba-Geigy);

2-hydroxy-2-methyl-1-phenyl-1-propanone, CAS Reg. No. 7473-98-5 (commercially available as DAROCUR® 1173 from Ciba-Geigy);

4-(2-hydroxyethoxy)phenyl]-2-(hydroxy-2-propyl)ketone, CAS Reg. No. 106797-53-9 (commercially available as IRGACURE® 2959 from Ciba-Geigy); and SR1129 photoinitiator, commercially available from Sartomer (Exton, Pa.).

Other suitable photinitiators can be used to practice the invention. See, for example, Anon., Photoinitiators for UV Curing: Key Products Selection Guide, Ciba Specialty Chemicals, Basel, Switzerland, 2002; Misev et al., Weather Stabilization and Pigmentation of UV-Curable Powder Coatings, Journal of Coatings Technology, issue of July/August, pages 34-41, 1999; and references cited in these references.

The initiators used in the present invention are preferably water soluble and may be mixed directly with the aqueous monomer solution in an amount of from about 0.1 μM to about 250 μM, that is, by way of non-limiting example, from about 0.5 μM to about 50 μM, from about 0.5 μM to about 25 μM, from about 1 μM to about 10 μM, from about 0.1 μM to about 10 μM, from about 0.5 μM to about 5 μM, about 0.1 μM, about 0.2 μM, about 0.5 μM, about 0.75 μM, about 1 μM, about 2 μM, about 5 μM, about 7.5 μM, about 10 μM, about 15 μM, about 25 μM, about 40 μM, about 50 μM, about 60 μM, about 75 μM, about 90 μM, about 100 μM, about 125 μM, about 150 μM, about 175 μM, about 190 μM or about 200 μM.

In these embodiments, the polymerization of the monomer solution is achieved by irradiating the solution with ultraviolet light. Any light source that will activate the initiators may be used. Preferred are light sources emitting light with a wavelength within from about 100 nm to about 500 nm. That is, by way of non-limiting example, from about 100 nm to about 500 nm, from about 150 nm to about 450 nm, from about 200 nm to about 400 nm, from about 300 nm to about 400 nm, from about 300 nm to about 450 nm, or from about 300 nm to about 500 nm.

A suitable amount of irradiation is generally from about 0.1 joule/cm² to about 100 joule/cm², that is, by way of non-limiting example, from about 0.2 joule/cm² to about 100 joule/cm², from about 0.1 joule/cm² to about 75 joule/cm², from about 0.5 joule/cm² to about 75 joule/cm², from about 1 joule/cm² to about 50 joule/cm² from about 1 joule/cm² to about 25 joule/cm², or from about 0.5 joule/cm² to about 10 joule/cm².

The precise concentrations of the polymerization agents necessary for optimal polymerization can be readily determined through trial and error by the skilled artisan. Final concentrations of TEMED and APS, for example, are typically in the range of 0.06-0.12% (v/v) and 0.04-0.12% (w/v), respectively. Higher or lower concentrations may be used as desired under a given condition to speed up or slow down the polymerization process.

The acidic and basic solutions may comprise other agents.

For example, additional buffering agents may be present in these solutions. Such agents may, for example, stabilize the pH of the poured gradient during the polymerization step. Other agents, such as sorbitol or glycerol may be added to one or the other of the solutions to facilitate formation and/or stability of the gradient prior to and/or during the polymerization step. Any soluble agent that is not covalently incorporated into the polyacrylamide matrix during the polymerization may subsequently be removed from the polymerized gel by washing if so desired.

In yet other embodiments, agarose may be included.

### Gel Casting

The polymerized acrylamide matrix of the gel and strips of the current invention may be cast by a variety of methods known to those of skill in the art, either between supporting plates of a cassette or exposed upon a support.

For example, IPG slab gels with linear pH gradients may be cast as described in Görg et al. (1986) Electrophoresis '86, Dunn, ed., VCH Weinheim, 435-449. Methods of casting IPG gels are also described in Righetti (1990) Immobilized pH gradients. Theory and methodology, Elsevier, Amsterdam, New York, Oxford, pp. 127-134. In these methods, one of the solutions contains an agent such as sorbitol or glycerol to increase its density, so that the poured gradient resists remixing. The gradient is formed using a two-vessel gradient maker connected by a capillary tube to a polymerization cassette. The gradient maker is placed on a magnetic stirrer, and the polymerization agents are added to each solution shortly before the gradient is poured. The gradient may be delivered either linearly or non-linearly as desired.

The gradient may alternatively be formed by precision pumps and/or burettes that deliver the desired volumes of each of the individual component solutions of the gel to a mixer prior to delivery of the mixed solution into a polymerization cassette. In such a pumping system, the polymerization agents may either be added directly to the acidic and basic solutions, or they may be pumped separately into the mixing chamber.

Software can be used to control the pumps and/or burettes. (Available from, e.g., Serva, Heidelberg, Germany.) Parameters are entered into the software program. The software is loaded into the memory of a computer connected to a series of precise pumps and/or burettes, wherein the software directs the dispensing of gel casting solutions by the precise pumps and/or burettes. The precision of the pumps and/or burettes in certain aspects is sufficiently high to achieve less 0.1% deviation of a dosing volume.

In other embodiments, the casting may be performed in a casting fixture made to orient a thin support film on a backplate. The casting fixture also provides a cavity of, for example, about 0.5 mm in which the acrylamide matrix is polymerized onto the support.

In one embodiment, the gels are cast vertically onto a thin support film held between two plates by spacers.

In a preferred embodiment, the thin support film is treated with vinyl moieties with which the acrylamide can polymerize and thereby cause the gel to adhere to the backing. Such thin film support is available commercially as GelBond® PAG film (Cambrex, East Rutherford, N.J., USA).

Following the casting in the casting fixture, the acrylamide is allowed to polymerize. In a preferred embodiment of the invention, the casting fixture is held at room temperature for 30 minutes and then at 50° C. for 1 hour in an oven. As indicated above, in other embodiments, a gradient maker can used to cast the IPG gels. IPG gels having the desired resolution and rehydration behavior and with precise pH ranges of 4.5-5.5 and 5.3-6.3 can been cast using the same acidic and basic solutions developed for the pH 4-7 strips and having the compositions shown in Table 4. Prior to casting, the addition of APS and TEMED to final concentrations of 0.1% from 1.56% stocks is necessary to provide polymerization after delivery of solutions is complete.

In order to cast the pH 4.5-5.5 IPG gels, a gradient maker may be used (available from Amersham Biosciences or VWR) with 5 mL of acidic solution and 5 mL of basic solution in their respective chambers. Manual delivery of 0.694 mL of acidic solution and 0.216 mL basic solution to the gel cassette may be required before allowing solution delivery from the gradient maker. The remaining solutions from the gradient maker may then be allowed to flow into the gel cassette. Different size gels may be cast by scaling the total volume of gel solutions delivered.

In order to cast the pH 5.3-6.3 IPG gels, a gradient maker may be used (available from Amersham Biosciences or VWR) with 5 mL of acidic solution and 5 mL of basic solution in their respective chambers. Manual delivery of 0.512 mL of acidic solution and 0.398 mL basic solution to the gel cassette may be required before allowing solution delivery from the gradient maker. The remaining solutions from the gradient maker may then be allowed to flow into the gel cassette. Different size gels may be cast by scaling the total volume of gel solutions delivered.

In order to cast the pH 6.1-7.1 IPG gels, a gradient maker may be used (available from Amersham Biosciences or VWR) with 5 mL of acidic solution and 5 mL of basic solution in the respective chamber. Delivery may be initiated from the gradient maker into the gel cassette and continued until all solution is delivered. Different size gels may be cast by scaling the total volume of gel solutions delivered.

### Gel Processing

After polymerization, gels and strips of the present invention may be washed; washing may usefully reduce contaminants, such as unpolymerized monomers, buffer, or catalyst.

In typical embodiments, the washing step is performed with deionized water. In a preferred embodiment, the gels are washed with a plurality of changes of wash water, such as 2, 3, 4, 5, even 6 or more changes of wash water, for at last about 5 minutes, 10 minutes, even 15 minutes each.

In yet other embodiments, the gels and strips of the present invention may be washed in a low ionic strength solution buffered near neutrality. The wash solution may conveniently be based on the low ionic strength buffers described in U.S. Pat. Nos. 5,578,180, 5,922,185, 6,059,948, 6,096,182, 6,143,154, 6,162,338, the disclosures of which are incorporated herein by reference in their entireties.

For example, the wash solution may comprise Bis-Tris((2-hydroxyethyl)iminotris(hydroxymethyl) methane), Tricine, glycerol and/or sorbitol, EDTA, sodium azide, and SB-14 (3-(N,N-dimethylmyristylammonio) propanesulfonate), titrated to a neutral pH.

In addition or in the alternative, the gels and strips of the present invention may be washed with one or more reducing agents, such as those included in the running buffers described, e.g., in U.S. Pat. No. 5,578,180, the disclosure of which is incorporated herein by reference in its entirety. The reducing agent may, e.g., be sodium bisulfite.

Usefully, the gels or strips of the present invention may thereafter be dried (dehydrated); such dehydration is not required, however. As further described below, if the gel is dehydrated, it must be rehydrated before isoelectric focusing therein.

Neither complete removal of moisture, during dehydration, nor complete saturation with liquid, during rehydration, is required or intended. It suffices for practice of the present invention that the gel or strip, if dehydrated, swell detectably after contact in its dehydrated state with an aqueous solution (“aqueous buffer”, “buffer”).

Typically, the dehydrated gel or strip will swell at least about 5% in volume, often at least about 10%, 15%, 20%, even at least about 25%, 30%, 40% or more in volume upon contact with an aqueous buffer. The volume increase may be manifest in all three dimensions or, when the gel matrix is cast upon an inextensible support, principally in one or in two dimensions.

Usefully, the degree of swelling is sufficient as to permit hydratable lodging in an enclosing member, such as an IPGRunner™ cassette (Invitrogen Corp., Carlsbad, Calif.).

In one exemplary method of drying, the gels and strips are incubated in glycerol and air-dried. Alternatively, the gels may be dried in a covered box with small computer fans to provide air circulation over the gels. In a preferred embodiment, the gels are dried overnight. In such embodiments, the gels are covered after drying with a polyester film and cut into strips for use in IEF.

### Gel and Strip Rehydration

In another aspect, the invention provides methods for analyzing proteins by isoelectric focusing (IEF) within the gel matrix of gels and strips of the present invention.

Dehydrated gel and strip embodiments of the present invention must be rehydrated, however, prior to their use in isolectric focusing (IEF) applications. In one embodiment, the gels and strips are rehydrated at room temperature in a solution containing urea, a detergent, DTT, ZOOM® Carrier Ampholytes of an appropriate pH range (Invitrogen, Carlsbad, Calif.) or their equivalent, and a dye such as bromophenol blue.

In illustrative embodiments, the strips are rehydrated in a solution that also contains a protein sample to be analyzed.

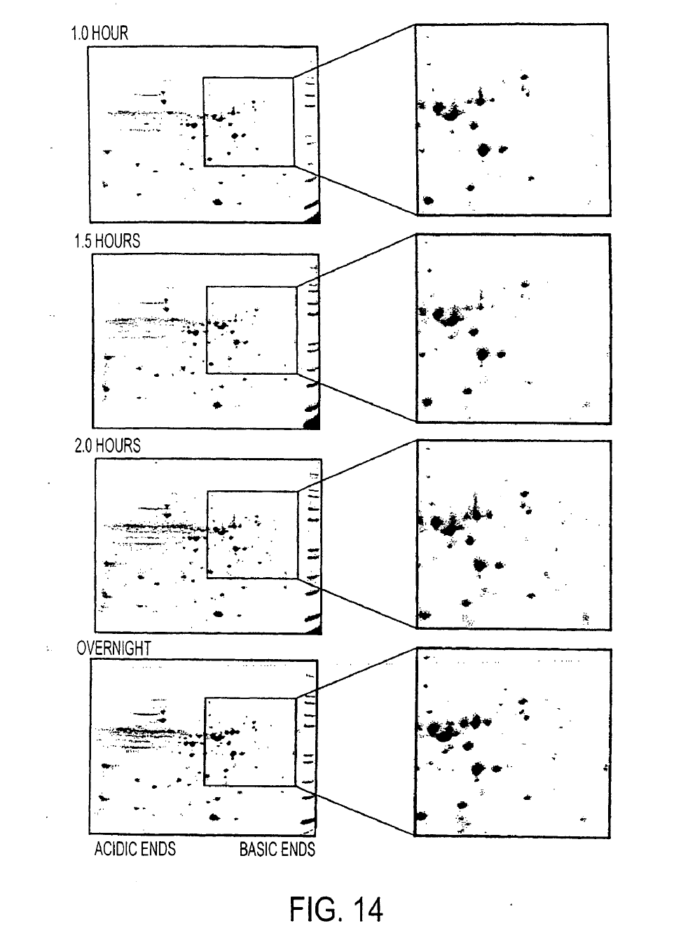

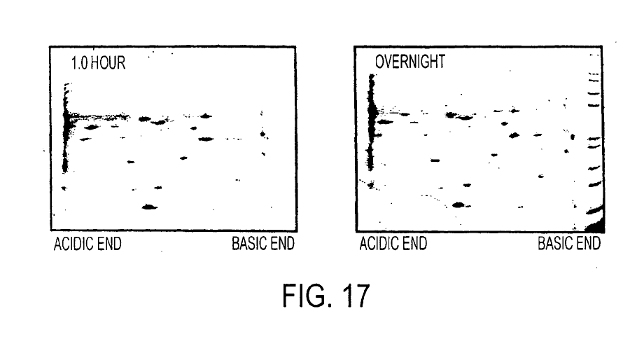

The novel gel matrix compositions of the present invention rehydrate more rapidly than do IPG strips known in the art. A dehydrated gel is rehydrated when the gel is capable of separating (i.e. focusing) molecules, typically proteins, according to their isoelectric point, from a sample contacted with the gel, such that a spot pattern achieved after the rehydrated gel with separated proteins is electrophoresed on a second dimension and stained, is similar, to the spot pattern achieved using the same 2-D electrophoresis method, except that the gel is allowed to rehydrate overnight. FIGS. 14 and 17 provide examples of the similarity in 2D spot pattern attained from an IEF gel strip that was successfully rehydrated after 1 hour compared to an IEF gel strip rehydrated overnight. Typically, the run to run reproducibility in spot patterns with a successfully rapidly rehydrated gel strip, is the same as the run to run reproducibility between the rapidly rehydrated gel strip and a gel strip that is rehydrated using an overnight rehydration (i.e. typically at least 10 hours, and in certain examples at least 12 hours).

In one embodiment of the invention, therefore, dehydrated gels and strips of the present invention are allowed to rehydrate for no more than 8 hours. In another embodiment, dehydrated gels and strips of the present invention are allowed to rehydrate for no more than 4 hours. In another embodiment, dehydrated gels and strips of the present invention are allowed to rehydrate for no more than 6 hours. Rapidly rehydratable gels and gel strips are gels and gel strips that successfully rehydrate in 6 hours, although as indicated herein the gel strips can typically rehydrate in 2 hours, 1 hour, or even 30 minutes.

In a preferred embodiment, dehydrated gels and strips of the present invention are allowed to rehydrate for no more than 2 hours, 90 minutes, or even no more than 60 minutes. In some embodiments, dehydrated gels or strips of the present invention are allowed to rehydrate for no more than 30 minutes, 15 minutes, 5 minutes, 1 minute, or even no more than 30 seconds. The speed of rehydration may be readily assessed, for example by comparison of the separation patterns for two identical samples run in gels or strips allowed to rehydrate for various times. See, e.g., Example 3 below.

Furthermore, the time of rehydration can be determined by determining the increase in mass or volume of a dried strip in the presence of a rehydration solution. The rehydration rate of a strip being tested can be compared to the rehydration rate of a dried gel strip such as a Zoom gel strip (Invitrogen, Calsbad, Calif.), for example by measuring a volume or mass increase in the gel strip over time. In certain aspects, if a strip being tested rehydrates in no more than 4, 3, 2, 1 hour, 30 minutes, 15 minutes, or 0 minutes more than a Zoom gel strip, the strip being tested is a rapidly rehydrated strip. Alternatively, completion of rehydration can be determined by identifying a timepoint at which a mass gain from rehydration achieves 90% of the mass gain it would have had from the classical overnight rehydration of 12 hours. In general, gel strips provided herein can be rehydrated such that within 1 hour of contact with a rehydration buffer, proteins can be separated according to isoelectric point within the gel strip.

When inserted into an IPGRunner (Invitrogen, Carlsbad, Calif.) along with 145 to 155 ul of rehydration buffer, gel strips of the present invention, within 1 hour of contact with the rehydration buffer, rehydrate to at least 90% of the mass volume attained after 16 hours of contact with the rehydration solution. In this particular example, approximately 125 microliters of rehydration buffer are absorbed by the gel strip within 1 hour of contact with the rehydration buffer. Typically, rehydration is performed at room temperature, however other temperatures can be used provided that rehydration can occur at these other temperatures.

### Methods of Using the Gels and Strips

The gels and strips of the present invention can be used to separate two or more molecules, or analytes, from each other, the method comprising electrophoresing a sample comprising the two or more molecules through the gel matrix. In embodiments in which the gel or strip has a fixed linear or nonlinear pH range, the method can include isoelectric focusing of the molecules within the gel matrix.

In embodiments in which the gel or gel matrix of a strip of the present invention is dry, the method typically comprises the antecedent step of rehydrating the gel matrix. Rehydration may be effected with the analytes in admixture with the rehydration buffer. The samples electrophoresed through the gel matrix in the methods of the present invention may usefully be chemically reduced during or prior to the electrophoresis. In some embodiments, the solution used to rehydrate the gel matrix comprises a chemical reducing agent. In other embodiments, the sample is reduced prior to contact with the gel matrix. In a preferred embodiment, the reducing agent is present during the electrophoresis. In another preferred embodiment, the reducing agent is 2-mercaptoethanol, tributylphosphine, or trishydroxyethylphosphine. In an even more preferred embodiment, the reducing agent is hydroxyethyldisulfide. See, e.g., Olsson et al. (2002) Proteomics 2:1630-32.

In some embodiments, the method may further comprise electrophoresing the sample in a second dimension, such as in a polyacrylamide gel that separates the analytes by size.

Accordingly, in one aspect, provided herein is a method for separating proteins of a sample using an electrophoretic field, that includes

a) rehydrating a dried gel strip; and

b) isoelectrically focusing proteins of the sample within the rehydrated gel strip, wherein the method is performed in no more than 2, 3, 4, 5, 6, 7 or 8 hours. In certain illustrative examples, the method is performed in no more than 5, 4, or 3 hours.

The method can further include placing the rehydrated gel comprising the isoelectrically focused proteins on a slab gel, and then separating the proteins in a second dimension according to a characteristic other than isoelectric point, wherein the method is completed in no more than 4, 5, 6, 7, or 8 hours. For example the method can be completed in about 4 hours. Before running the second dimension the rehydrated gels comprising the isoelectrically focused proteins is typically equilibrated in a buffer, for example a sample buffer for performing the second dimension, such as SDS-PAGE sample buffer. The equilibration can be performed for example, in 15 minutes, 25 minutes, 30 minutes, 35 minutes, 40 minutes, 45 minutes, 50 minutes, 55 minutes, 1 hour, or 2 hours.

The second dimension in methods of this aspect, can be any second dimension known in the art. In certain illustrative examples, in the second dimension proteins are separated based on their molecular weight.

The rehydrated gel strip used in the method is a rehydrated gel strip of the present invention. In illustrative examples, the rehydrated gel strip has a buffer capacity beta value of at least 3, for example 4 to 6 mEq/L/pH.

In a related aspect, the present invention provides a method for separating biomolecules of a sample, typically polypeptides or proteins of a sample, using gel electrophoresis, wherein the method includes rehydrating a dried gel strip; and separating one or more molecules, typically biomolecules, typically proteins, in the sample within the rehydrated gel strip based on the isoelectric point of the one or more molecules, wherein the method is completed in no more than 6, 5, 4, 3, or 2 hours. In illustrative examples, the method is completed in about 3 hours.

In certain examples, the method further includes placing the rehydrated gel comprising the separated one or more proteins on a slab gel, and electrophoresing the one more proteins into the slab gel, wherein the method is completed in no more than 10, 9, 8, 7, 6, 5, or 4 hours. In illustrative embodiments, the method is completed in about 4 hours.

In particularly useful embodiments, the sample is a biological sample, including a sample comprising proteins, and at least one of the analytes desired to be separated is a protein.

The analytes, such as proteins, may be visualized by contacting the gel with a compound that binds nonspecifically to proteins, such as a stain. Stains useful in electrophoresis are well known in the art and are available commercially. For example, kits for gel staining include the SimplyBlue™ SafeStain Colloidal Blue kit, SilverQuest™ kit and SilverXpress® kit from Invitrogen Corp. (Carlsbad, Calif., USA). Accordingly, in the illustrative aspects provided above, the method including rehydration, isoelectric focusing, second dimension gel electrophoresis, and staining is completed within 10, 9, 8, 7, 6, 5, or 4 hours.

Alternatively, proteins may be contacted with an agent that binds specifically to one or more protein analytes, such as an antibody or antigen-binding portion thereof directed to an epitope present in at least one protein in the protein sample, a chromogenic substrate of an enzyme in the protein sample, or a nucleic acid having a nucleotide sequence that is specifically bound by a nucleic acid binding protein in the protein sample.

The sample may comprise more than about 100 proteins, more than about 500 proteins, even more than about 1,000 proteins, wherein at least a plurality of the proteins may be variants selected from the group consisting of allelic variants, species markers, splicing variants, members of protein families, and species of post-translationally modified proteins.

### Kits

In another aspect, provided herein is a kit that includes one or more hydratable gel strips of the invention. In certain examples, the kit includes 2, 3, 4, 5, 6, 7, 8, 9, 10, 11, 12, 13, 14, 15, 16, 17, 18, 19, or 20 gel strips. The strip can be labeled with a unique identifying number, pH range, and/or an orientation mark. The gel strips can be supplied attached to a tri-fold card to help facilitate access and removal. In certain examples, the kit can include an apparatus for performing isoelectric focusing, for example the ZOOM IPGRunner Mini-Cell (Invitrogen, Carlsbad, Calif.). The kit can also include a rehydrating buffer and instructions for rehydrating the gel strip of the kit within 30 minutes, 1 hour, 2 hours, 3 hours, 4 hours, 5 hours, 6 hours, 7 hours, or 8 hours. In certain aspects, the kit can include one or more SDS PAGE slab gels.

### Methods for Selling Hydratable Gel Strips

In another aspect, provided herein is a method for selling a plurality of hydratable gel strips for isoelectric focusing, that includes advertising that the hydratable gel strips are rehydratable in no more than 6, 5, 4, 3, 2, or 1 hour, or 45 or 30 minutes. In certain examples, it is advertised that the hydratable gel strips are rehydratable in no more than 1 hour. The plurality of gel strips can include, for example, 2 to 40 gel strips. In another aspect, only 1 gel strip at a time is advertised and/or sold. The method typically includes an order entry function by which a customer can order the hydratable gel strips from a provider. For example, the order entry function can include functionality for accepting orders for the hydratable gel strips, or kits containing the hydratable gel strips, over a phone system, a facsimile system, and/or a computer network, such as a wide-area network, for example the Internet.

The advertising can take any form known in the advertising arts, including print advertising, such as printed manuals or advertisements in scientific journals or other trade publications, on-line advertising, such as on-line access to brochures and/or manuals, or advertising at a conference for example using materials and/or information available from a booth.

In certain aspects, the advertising includes displaying images of experiments performed using gel strips that were rehydrated in no more than 4 hours.

In another aspect, provided herein is a method for selling an electrophoresis system that utilizes hydratable gel strips for isoelectric focusing, comprising advertising that the electrophoresis system can be used to perform 2-D gel electrophoresis in no more than 12, 11, 10, 9, 8, 7, 6, 5, or 4 hours.

The following examples are offered by way of illustration, not by way of limitation.

### Example 1

### Use of IPG Strips and the ZOOM® IPGRunner™ System

The speed and ease of using the IPG strips of the present invention in a method provided herein in the ZOOM® IPGRunner™ System are summarized in Table 3. Further information regarding this use is incorporated by reference to the ZOOM® IPGRunner™ System instruction manual (Version C, Mar. 11, 2003, Invitrogen, Carlsbad, Calif.), which is incorporated herein by reference in its entirety.

TABLE 3
Fast proteomics results using the ZOOM ® IPGRunner ™
System

| Step | Procedure | Time | |
|---|---|---|---|
| 1 | Apply sample, insert IPG strips and seal | 10 | min. |
| | loading wells | | |
| 2 | Rehydrate IPG strips | 60 | min. |
| 3 | Remove wells, apply wicks and assemble | 5-20 | min. |
| | the ZOOM ® IPGRunner ™ Mini-cell | | |
| 4 | Perform isoelectric focusing | 90 | min. |
| 5 | Reduce (15 min.), alkylate (15 min.) and | 35 | min. |
| | insert IPG strip into a ZOOM ® Gel | | |
| 6 | Perform SDS PAGE | 40 | min. |
| 7 | Stain gel using SilverQuest ™ Silver | 90 or 45 | min. |
| | Staining Kit or SimplyBlue ™ SafeStain | | |

### Example 2

### Production and Use of Novel IPG Strips

### Materials

### Protein Standards

Protein standard solutions were made for evaluation in both the first and the second dimension under denaturing conditions. Various protein blends were used to evaluate the IPG strips. The blends included, for example, 640 μg/mL each of soybean trypsin inhibitor, carbonic anhydrase from bovine or human erythrocytes, actin from bovine muscle, bovine serum albumin, and lysozyme from chicken egg white. Lyophilized proteins were dissolved in water and then subsequently added to sample rehydration buffer containing 8M Urea, 2% CHAPS, ZOOM® Carrier Ampholytes (Invitrogen, Carlsbad, Calif.) (at concentrations indicated in experiments) and trace bromophenol blue. The concentration of each protein was approximately 70 μg/mL or ˜10.8 μg loaded per strip.

### Gel Casting

Gels were cast using a precision pumping system that delivers the desired volumes of the solutions and mixes the solutions just prior to their transfer into the casting fixture. The specific volumes of each buffer solution were determined using a software program to perform standard calculations after entering into the program, buffer compositions for the acidic and basic buffers and a desired pH gradient range. For gels having pH range 4-7, the solutions described in Table 4 were loaded in the pumping system, together with separate reservoirs of TEMED, 40% APS, and water.

### Sample Rehydration Buffer

The solution used to rehydrate samples contained: 8 M urea, 2% w/v CHAPS, 20 mM DTT, 0.5% v/v ZOOM® Carrier Ampholytes 4-7 (Invitrogen, Carlsbad, Calif.), and 0.0025-0.005% w/v bromophenol blue. A 9 M urea stock solution was deionized using, for example, AG 501-X8(D) resin (Bio-Rad, Hercules, Calif.) according to supplier instructions prior to formation of the solution. The resin slurry was passed through a 0.2 micron syringe filter. The resulting volume was measured to determine the amount of additional ultrapure water needed for achieving a final urea concentration of 8 M. CHAPS, DTT, ampholytes, and bromophenol blue were then added to achieve the indicated concentrations. The prepared solution was stored in a freezer in 1.8 mL aliquots.

### Solutions for Preparing Focused Strips for the 2nd Dimension Gel

After samples had undergone isoelectric focusing in the first dimension, IPG strips were incubated in a reducing solution and then in an alkylating solution prior to being loaded on the second dimension gel. Alkylation of the cysteine residues on proteins following reduction of the disulfide bonds reduces vertical streaking in the second dimension. The reducing solution contained 50 mM DTT in 1× NuPAGE® LDS Sample Buffer (Invitrogen, Carlsbad, Calif.). The alkylating solution contained 125 mM iodoacetamide in 1× NuPAGE® LDS Sample Buffer (Invitrogen, Carlsbad, Calif.).

### Methods

### Isoelectric Focusing Protocol

Isoelectric focusing was performed as generally described in the ZOOM® IPGRunner™ System instruction manual, incorporated herein as Appendix A. See also, U.S. Patent Publication No. 2003/0015426, which is hereby incorporated by reference in its entirety. A sample volume of 155 μL was used in all experiments unless otherwise indicated.

Following rehydration, the sample loading wells were removed. When necessary, the gels were adjusted to a position where both the acidic and basic ends were exposed in the window of the film in order to make contact with the electrode wicks. Electrode wicks were applied and wetted with 750 μL of deionized water. The ZOOM® IPGRunner™ Core and the ZOOM® IPGRunner™ Cassette were assembled according to instructions. When a single cassette was used, a buffer dam was put in place of the second ZOOM® IPGRunner™ Cassette. The outer buffer chamber was filled with approximately 650 mL of deionized water. No water was placed in the inner chamber.

Except as otherwise indicated, isoelectric focusing was performed using a programmable power supply with a 50 μA/strip current limit with the following voltage steps:

175 V for 15 minutes

175-2000 V ramp for 45 minutes

2000 V for 1 hr 45 minutes

### SDS PAGE Protocol

Electrophoresis in the second dimension was performed on ZOOM® Gels (Invitrogen, Carlsbad, Calif.). If the second dimension was not performed immediately following the isoelectric focusing step, the IPG strips were stored in a sealed plastic bag at −80° C. until use.

Equilibration for the second dimension was carried out in two steps, with the first step containing SDS and reducing agent and the second step containing SDS and alkylating agent. The SDS in both steps prepares the IPG strips for second dimension SDS electrophoresis. In the reduction step, strips were incubated for 15 minutes in 5 mL of the Reducing Solution at room temperature. The Reducing Solution was decanted, and alkylation was performed by incubation for 15 minutes in 5 mL of the Alkylating Solution. Alkylation of the sulfhydryl groups of the proteins was performed to reduce vertical streaking. Excess iodoacetamide destroys residual DTT, which when present may cause vertical streaking. Agarose (0.5% w/v) was prepared in the appropriate SDS electrophoresis running buffer. The IPG strip was placed into the ZOOM® Gel well and sealed in the well with approximately 400 μl of the 0.5% w/v agarose solution. Molecular weight standards were loaded in the molecular weight marker well. Gels used for the electrophoresis in the second dimension were either NuPAGE® 4-12% Bis-Tris ZOOM® gels or Novex® 4-20% Tris-Glycine ZOOM® gels with the XCell SureLock™ Mini-Cell according to the standard protocol. Electrophoresis was performed at 200 V for 35-50 minutes for NuPAGE® Novex® Bis-Tris ZOOM® gels or at 125 V for 90 minutes for Novex® Tris-Glycine ZOOM® gels.

### Gel Staining

When desired, strips run in the first dimension may be stained for 30 minutes with a Coomassie®-R250/cupric sulfate stain containing 0.50 g/L Serva Blue R, 1.71 g/L copper sulfate pentahydrate, 25% v/v ethanol, and 19% v/v glacial acetic acid. After the staining period, the strips are washed several times with wash buffer containing 30% ethanol and 7% glacial acetic acid until the background is clear.

Gels run in the second dimension are stained with SilverQuest™ or SimplyBlue™ SafeStain kits (Invitrogen, Carlsbad, Calif.) according to the manufacturer's instructions.

### Image Analysis

Band migration may be analyzed by using alignment grid overlays to determine if proteins have migrated to their proper location. Strips are scanned on a glass scanning alignment grid with a UMAX PowerLook III scanner using Magic-Scan 32V4.5 software. The strip image is then pasted into an Excel file containing the appropriate overlay grid. The overlay is then used to evaluate whether or not the protein standards migrated to the correct position on the narrow pH range IPG strips.

### Stability Testing

IPG strips of each pH range were placed in foil pouches at 37° C., 25° C., 4° C., −20° C., and −80° C. At each time point, two strips from each gel were allowed to come to room temperature before side-by-side rehydration overnight. One strip was rehydrated with an E. coli lysate sample and the other with a protein standard that includes proteins with pI values within the pH range of the strip. Both strips were subjected to IEF as described above.

### Determination of Run Parameters for Narrow pH Range IEF Gel Strips

IPG strips were run on the ZOOM® IPGRunner Apparatus using a programmable power supply. IPG strips were rehydrated using 155 μL of 8 M urea, 2% w/v CHAPS, 20 mM DTT, 0.5% v/v ZOOM® Carrier Ampholytes 4-7 (Invitrogen, Carlsbad, Calif.) and a protein standard blend. Strips were focused with one of each narrow pH range strip in the same cassette, three strips to a cassette. Cassettes were focused as described above, except that no current or watt limits are used, and the final 2000V step was held for 45, 65, 85, 105, or 120 minutes. After focusing, the strips were analyzed in the second dimension by SDS-PAGE as described above.

### Effect of Ampholyte Type and Concentration on IEF

Protein standard and E. coli lysate samples were run on the IPG strips with varying concentrations and compositions of ampholytes (detailed below in Results and Discussion). Briefly, broad pH range ampholytes (pH 3-10 and 4-7), narrow pH range ampholytes (pH 4-6, 5-7, and 6-8), and blends of broad and narrow pH range ampholytes were used at varied concentrations in place of the standard 0.5% v/v ZOOM® Carrier Ampholytes 4-7 (Invitrogen, Carlsbad, Calif.) in the Sample Rehydration Buffer. Sample loads were 10-20 μg of the protein standard, while lysate loads were either 10 μg for silver stain or 100-200 μg for Coomassie stain. Stained SDS-PAGE gels were evaluated for completeness of focusing by visual inspection.

### Results and Discussion

### Formula Development for pH 4-7 Strips

In order to decrease the streaking of protein spots in 2DE and to reduce the time necessary to rehydrate IPG strips prior to IEF, a novel formula containing higher concentrations of acrylamido buffers in the polymerized acrylamide matrix was developed. This solution contains the volumes of each of the components shown in Table 4.

TABLE 4
Composition of Acidic and Basic Solutions Used in IPG
Gels

| | Amount in | | Amount in | |
|---|---|---|---|---|
| | acidic | | basic | |
| Component stock | solution | | solution | |
| pKₐ 3.6 Acrylamido buffer | 23.1225 | mL | 12.0825 | mL |
| (0.2 M) | | | | |
| pKₐ 4.6 Acrylamido buffer | 4.4025 | mL | 29.520 | mL |
| (0.2 M) | | | | |
| pKₐ 6.2 Acrylamido buffer | 18.000 | mL | 6.0375 | mL |
| (0.2 M) | | | | |
| pKₐ 7.0 Acrylamido buffer | 0 | | 10.7625 | mL |
| (0.2 M) | | | | |
| pKₐ 9.3 Acrylamido buffer | 0 | | 35.040 | mL |
| (0.2 M) | | | | |
| Tris base (1 M) | 5.625 | mL | 0.3375 | mL |
| Acrylamid:bisacrylamide | 55.470 | mL | 55.470 | mL |
| (30%:0.93%) | | | | |
| Sorbitol (solid) | 103 | g | 0 | |

Each solution was adjusted to a final volume of 300 mL with ultrapure water. The above compositions provide for the proper final concentrations of all components following addition of APS and TEMED. Gels are cast from a linear gradient of the acidic solution and the basic solution to generate gels displaying a pH range from 4-7.

### Formula Development for Narrow-Range Strips

Improved properties in IEF with IPG strips displaying narrower pH ranges have also been achieved. These improved strips provide high resolution of proteins in the first dimension, migration properties consistent with those of the broader-range IPG strips, and rapid rehydration.

Initial 1 pH unit narrow-range IPG strips were cast using published formulas given by Righetti in Immobilized pH Gradients: Theory and Methodology, Table 2.2, pp. 64-67, which is hereby incorporated by reference in its entirety. Strips were cast using standard procedures. The performance of all strips was evaluated by staining first dimension and second dimension gels as described below. The migration of standard proteins, with known pI values, was evaluated and compared to the migration on commercial strips. Well-focused spots on SDS-PAGE gels and bands on IPG strips were observed, but gels cast using the Righetti-specified pH 4.5-5.5, 5.3-6.3, and 6.1-7.1 recipes did not give the expected protein migrations, and the expected 0.2 pH-unit overlap between these gels was not observed. Based on these results, additional gels were cast using published recipes having specified pH gradients outside our desired pH ranges. Analysis of protein migration on these gels indicated that the published formulas yielded a greater than 1 pH-unit range. New formulations were therefore developed and optimized to meet performance requirements for narrow pH range IPG strips with pH ranges of 4.5-5.5 and 5.3-6.3.

### Performance Evaluation

Over the course of development, performance of prototype strips was evaluated by IEF separation of known protein standards in the first dimension on Coomassie-stained IPG strips and in the second dimension on Coomassie or silver-stained SDS-PAGE gels. IPG strips stained following IEF were evaluated visually, and resolution was judged by position and shape of protein bands. SDS-PAGE gels were evaluated by visual assessment of protein spot position.

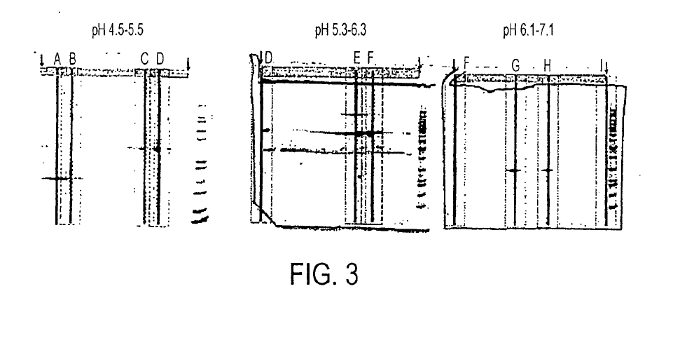

Band position was determined by alignment with an overlay grid that defines the expected position of the protein standards in the pH gradient being analyzed (FIG. 3). The overlay grid seen in FIG. 3 consists of solid lines representing the expected protein migration and the tolerances represented by the dashed lines on either side. The tolerances were taken from those used for broad range strips which in turn were derived from reproducibility studies and other sources. Soybean trypsin inhibitor (STI) and actin were used as standards for pH 4.5-5.5 IPG strips. Actin and bovine serum albumin (BSA) were used as standards for pH 5.3-6.3 IPG strips. Two different isoforms of carbonic anhydrase (CAI & CAII) were used as standards for analyzing pH 6.1-7.1 IPG strips. In each gel of FIG. 3, protein isoforms with pI's assigned by analyzing Invitrogen 4-7 strips have migrated to their expected location, indicating that each narrow pH range strip indeed has a pH gradient matching its designated pH range (4.5-5.5, 5.3-6.3, and 6.1-7.1).

In addition, measurement of the pH of the strips directly provides support that the desired pH gradient for each strip has been achieved and correlates with the performance evaluation described above.

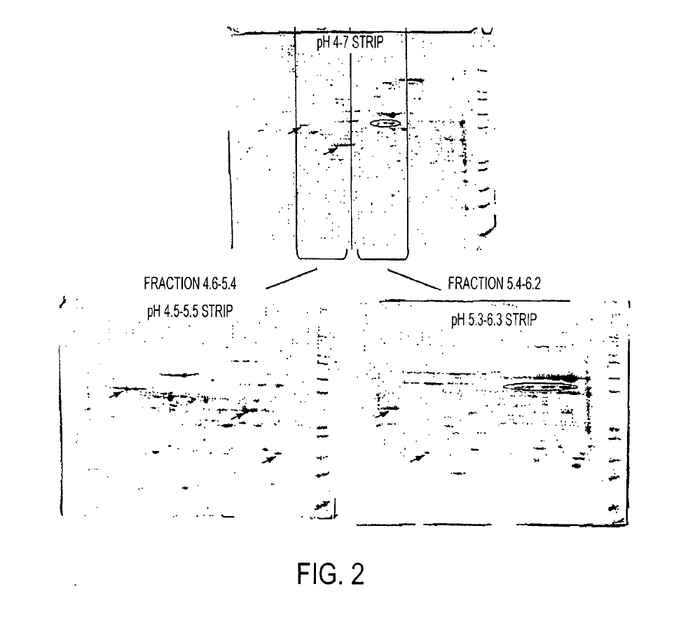

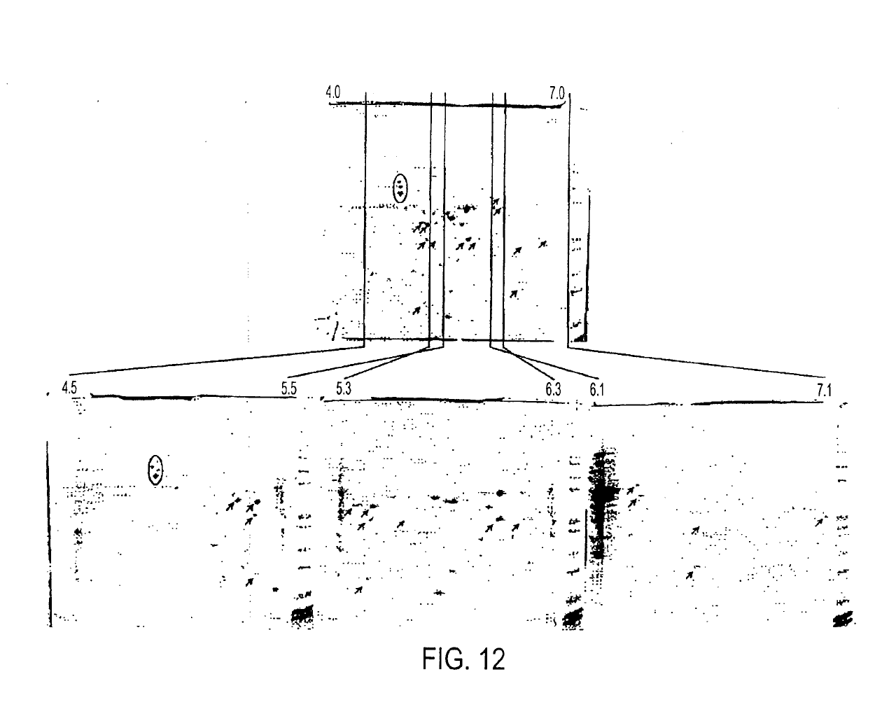

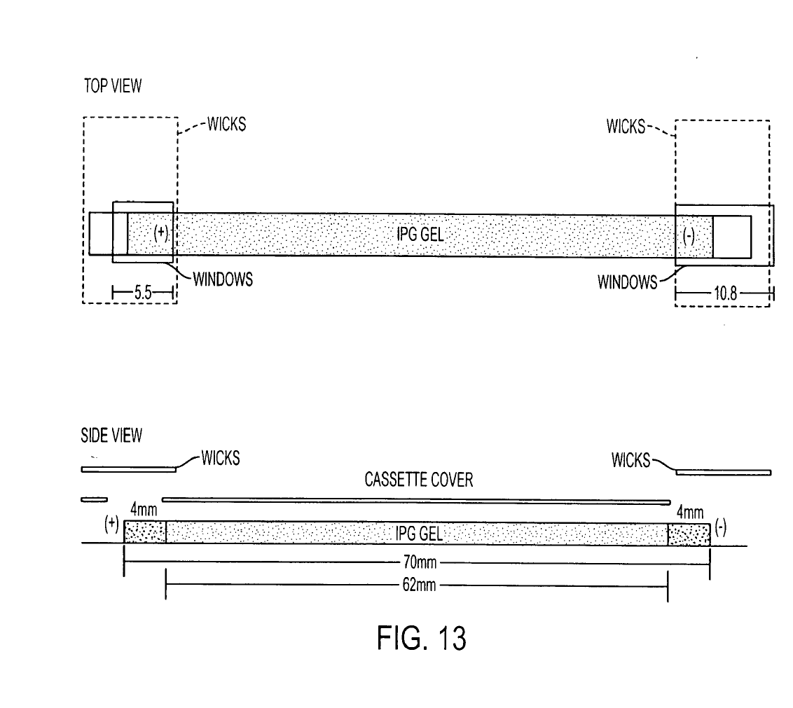

E. coli lysate and fractions from the ZOOM® IEF Fractionator (rat liver lysate and E. coli lysate) were also separated by 2DE using all three narrow pH range strips (FIGS. 2, 7-12). The demonstration of equivalent constellations of protein spots on 2D gels, when comparing narrow pH range gels to broad pH range gels (FIGS. 2 and 12), also indicates that the pH gradients in the narrow pH range strips meet the performance requirements stated above.

### Load Capacity

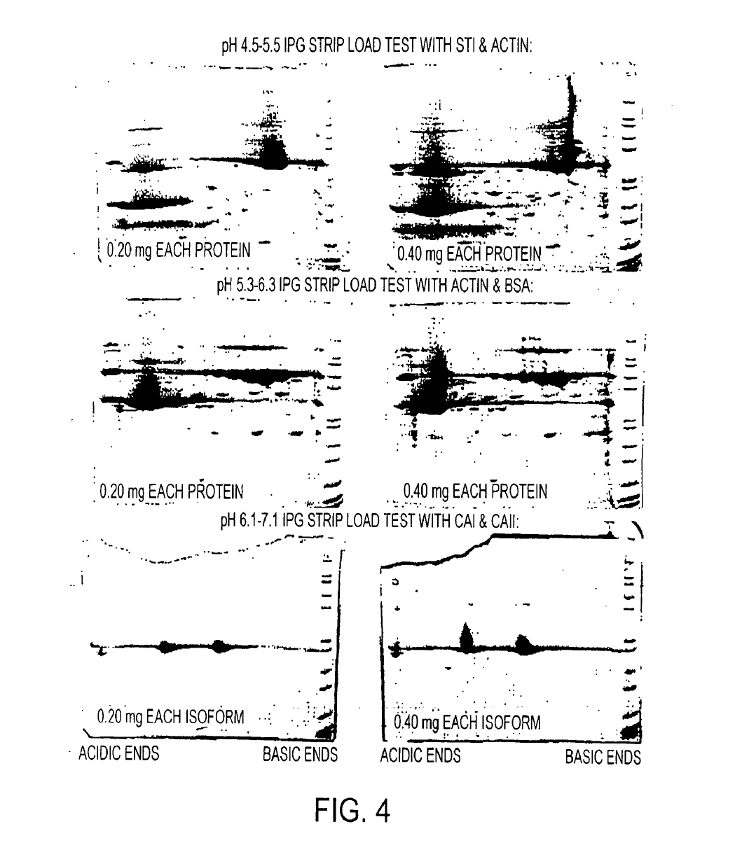

Higher proteins loads are needed in biological samples to detect and analyze proteins that are in low abundance in the tissue of interest. The amount of total protein that can be loaded on an IPG strip may be limited by the concentration of highly abundant proteins in a sample. The intent of loading high protein levels is not to test the behavior of highly abundant proteins (which could be analyzed at much lower loads) but rather to detect, identify, and analyze low abundance species which may be much more relevant biologically. Protein load capacity for each of the narrow IPG ranges was evaluated using the protein standards at 0.01, 0.05, 0.1, 0.2, and 0.4 mg loads. FIG. 4 shows 2D gels for each pH range. The low level contaminants in these highly purified proteins can be seen as well focused spots on all gels. The vertical and horizontal streaking of the proteins standards mimics what is typically seen in biological samples containing highly abundant proteins. These highly abundant proteins would resolve at lower loads as seen in FIG. 3. However, the goal in loading higher levels of sample is to detect and analyze proteins of biological significance that are generally in low abundance in the cell. A total protein load of 800 μg can result in minor components behaving in the system (i.e. resolved and detectable) as if they were the sole components of the load. The vertical streaking seen in the highly abundant proteins may be due to inefficient reduction and alkylation of the proteins due to their high concentration. The horizontal streaking of the highly abundant proteins may be due to inadequate focusing time. The focusing of minor components in the same gel is quite good, however (FIG. 4), illustrating the ability of the narrow pH range strips to “zoom” in on biologically relevant low abundance proteins even in the presence of extremely high amounts of abundant proteins. Horizontal streaking may also be observed when the total protein load exceeds the buffering capacity of the acrylamido buffers in the strip, with the result that proper focusing cannot be achieved. However, the focusing of low abundant proteins in the presence of the highly abundant proteins indicates that the buffering capacity of the narrow pH range IPG strips has not been exceeded (FIG. 4).

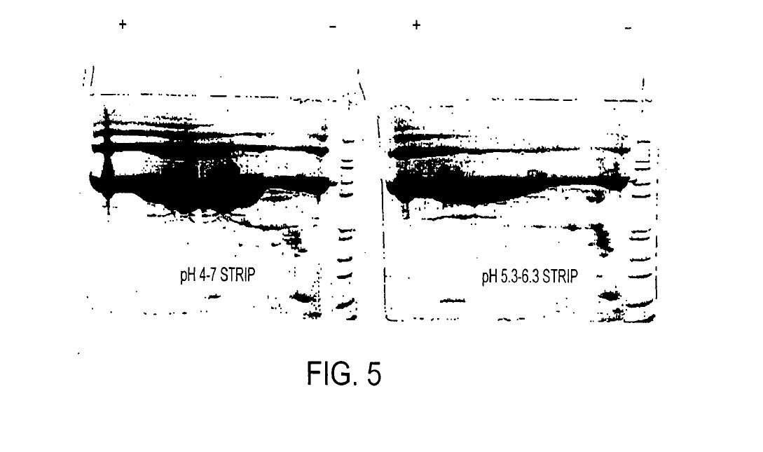

In addition, a pH 5.3-6.3 IPG strip was rehydrated with an extreme protein load of 7.75 mg of BSA. An identical sample was applied to a pH 4-7 IPG strip for comparison. The 2D gels are shown in FIG. 5. The degree of resolution at this loading capacity is surprisingly good considering the extreme range of the protein sample from minor to major components. This ability to resolve minor components in the presence of a few major species is a demonstration of the capabilities of these narrow range strips. The utility of this capability is shown in instances where abundant proteins are inseparable from the proteins of interest, as would be the case for minor serum proteins in the presence of serum albumin or immunoprecipitation analysis of proteins of interest where antibody is commonly highly abundant. FIGS. 4 and 5 illustrate this point with an extreme load (up to 7.75 mg) of serum albumin, a common abundant protein that limits protein load. Minor species are still detected and resolved in the gels shown. This does not of course address the issue of 2D co-localization of abundant and minor species, which can be addressed using complexity reduction, for example through the use of an additional fractionation step such as with the ZOOM® IEF Fractionator.

### Electrical Run Parameter Optimization

The run parameters for the narrow range strips were established by performing a time-course on the last step in the electrical protocol. Extended focusing requirements in terms of volt-hours are expected for the narrow range strips compared to the broader pH range strips. This is due to an increased resistivity as the proteins approach their pI with the smaller incremental change in pH over the 7 cm strip.

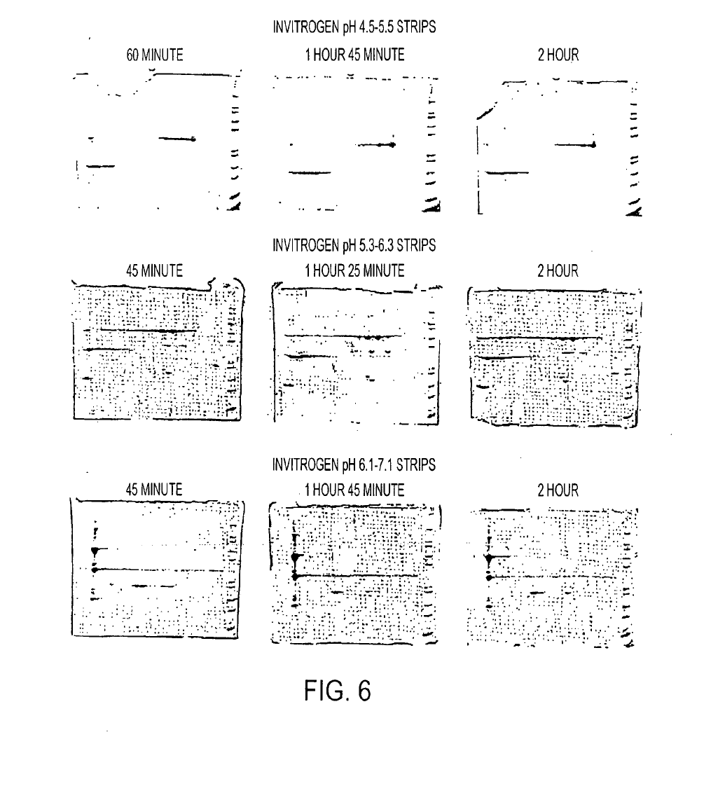

The three pH range strips were focused in separate cassettes for five different time durations. All five runs were focused for an initial step at 175V for 15 minutes followed by a ramp from 175V-2000V for 45 or 60 minutes. Following the ramp, the runs were held at 2000V for 45, 65, 85, 105, and 120 minutes. The 2D gels are shown in FIG. 6. The degree of sharpness of spots in the second dimension determines completion of the run. Spots showed little change in the degree of sharpness with a final 2000V step greater than 1 hour and 45 minutes. Isoforms of carbonic anhydrase were shown to form two distinct spots only after 1 hour and 45 minutes, while isoforms of albumin are nearly resolved at 1 hour and 25 minutes. The focusing appears to be complete for all sample components after the strips had been held at 2000V for 1 hour and 45 minutes (approximately 4200 volt-hours), and this is the recommended time for focusing. A time course run with a 100 μg load of standards results in comparable results. These parameters are used in subsequent experiments in which protein standards are compared to ZOOM® IEF Fractionator separated samples and crude lysates.

### Stability Testing

IPG strips produced according to the formulations disclosed herein display normal stability. Strips stored at −20° C. and 4° C. for 26 days show similar focusing and little, if any, decrease in protein absorption by the strips stored at 4° C. Strips stored at elevated temperatures for 26 days show a marked decrease in protein absorption when compared to strips stored at −20° C.; despite the decrease protein absorption, however, these strips retain the ability to properly focus the absorbed proteins.

### IEF on Narrow-Range IPG Strips of Samples Previously Focused by the ZOOM™ IEF Fractionator

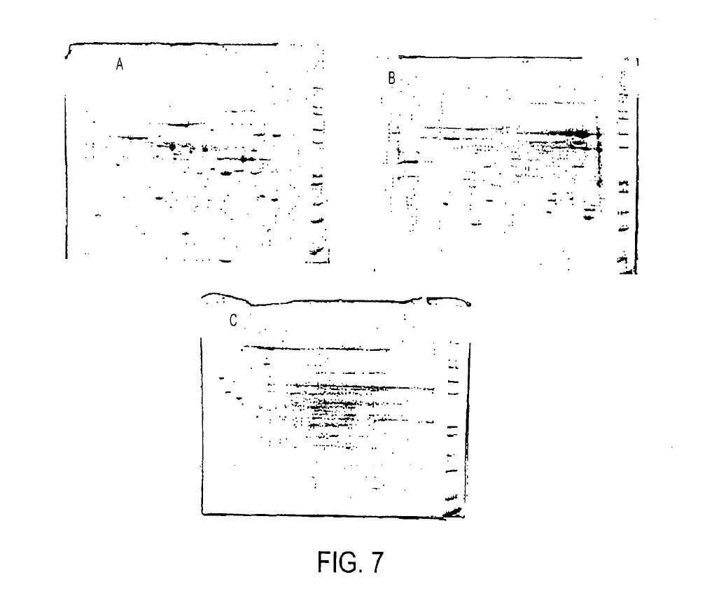

The ZOOM® IEF Fractionator (Invitrogen, Carlsbad, Calif.) may be used to fractionate protein samples in solution prior to their analysis by 2DE using IPG strips. Rat liver lysate was pre-fractionated with the ZOOM® IEF Fractionator device to yield fractions containing proteins with pI's in the ranges of 3.0-4.6, 4.6-5.4, 5.4-6.2, 6.2-7.0, and 7.0-10.0. The three middle fractions (4.6-5.4, 5.4-6.2, and 6.2-7.0) were further separated by 2D-PAGE using narrow pH range IPG strips that spanned the pH range of the ZOOM® IEF Fractionator fractions. The ZOOM® IEF Fractionator fraction 4.6-5.4 separated on 4.5-5.5 strips showed good focusing (FIG. 7A). The “cut-offs” for pre-fractionation appear to be close to the desired values. This is reflected in the absence of accumulation (“pile-up”) of protein on the acidic or basic side of the gel, which indicates that the pH range of the pre-fractionated sample fits well within the pH range of the strip (FIG. 7A). The 5.4-6.2 sample from ZOOM® IEF Fractionator separated on the 5.3-6.3 strip shows focused protein spots across the entire length of the gel, with a small basic-end “pile-up” (FIG. 7B). The pre-fractionated 6.2-7.0 sample separated on the 6.1-7.1 strip shows proteins across the pH range of the strip with very little “pile-up” at either end (FIG. 7C).

Pre-fractionated samples from the ZOOM® IEF Fractionator device applied directly to the strips contain 0.2% ZOOM® Carrier Ampholytes 3-10 (Invitrogen, Carlsbad, Calif.). The ampholyte requirement for separation on narrow range strips was examined on the three fractions containing the 0.2% ZOOM® Carrier Ampholytes 3-10 (Invitrogen, Carlsbad, Calif.) by adding either 0.5% ZOOM® Carrier Ampholytes 4-7 (Invitrogen, Carlsbad, Calif.) or 0.5% Servalytes (Serva) pH 4-6, 5-7, 5-8 corresponding to IPG strip 4.5-5.5, 5.3-6.3, 6.1-7.1 respectively. The ZOOM® IEF Fractionator fraction 4.6-5.4 separated on 4.5-5.5 strips displayed good focusing with all ampholyte mixtures used. ZOOM® IEF Fractionator fraction 5.4-6.2 separated on 5.3-6.3 strips showed improved focusing with the addition of 0.5% ZOOM® Carrier Ampholytes 4-7 (Invitrogen, Carlsbad, Calif.) to the 0.2% ZOOM® Carrier Ampholytes 3-10 (Invitrogen, Carlsbad, Calif.). ZOOM® IEF Fractionator fraction 6.2-7.0 separated on 6.1-7.1 strips showed a significant change in focusing with a change in ampholyte type and concentration. The addition of either 0.5% Servalytes pH 6-7 or 1.0% ZOOM® Carrier Ampholytes 4-7 improved focusing over using 0.2% ZOOM® Carrier Ampholytes 3-10 alone. Based on these observations a new set of narrow pH range ZOOM® Carrier Ampholytes with pH ranges of 4-6, 5-7 and 6-8 were incorporated into the run parameters.

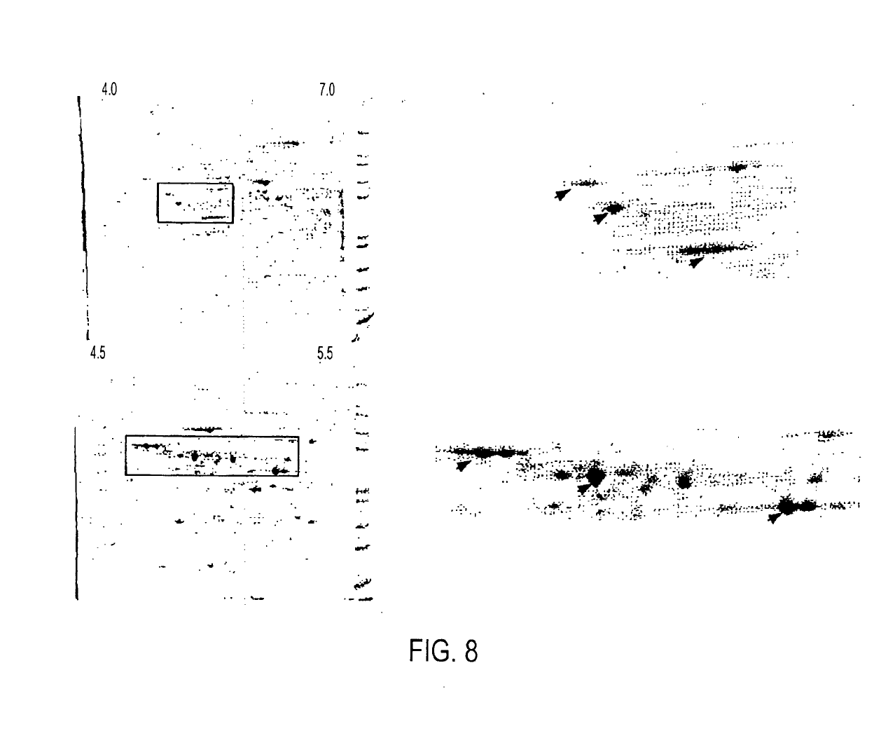

A comparison of rat liver lysate samples separated by 2D-PAGE with or without pre-fractionation revealed the increase in resolution provided by ZOOM® IEF Fractionator pre-fractionation using the narrow range 4.5-5.5 IPG strip (FIG. 8). The “zoomed-in” portion of the gels reveals more tightly focused spots and better resolution of protein isoforms in the pre-fractionated sample.

### Lysate Samples Focused on Narrow pH Range IPG Strips

Whole lysate samples provide a more complex protein mixture and; therefore, a more challenging sample for the narrow pH range focusing technique. Part of the challenge is that only a portion of the lysate sample will focus within the window provided by the narrow pH range IPG strip, leaving the remainder of the lysate to pile up at the electrodes. This means that only a portion of the total protein loaded onto the strip will be visualized as focused proteins, which decreases the sensitivity of the method. This can be alleviated by either loading much more protein lysate onto the strip or by pre-fractionating, thereby concentrating the desired protein in the sample. There are many methods for pre-fractionating or decreasing the complexity of a protein sample, but for those who would like to run lysates as a “first-try” method for protein isolation, narrow pH range IPG strips have been tested with E. coli lysate.

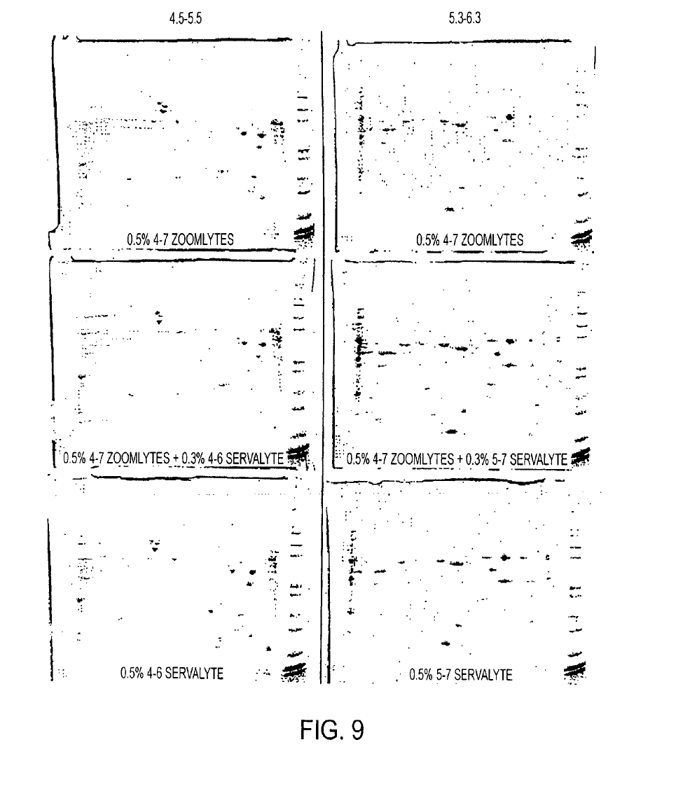

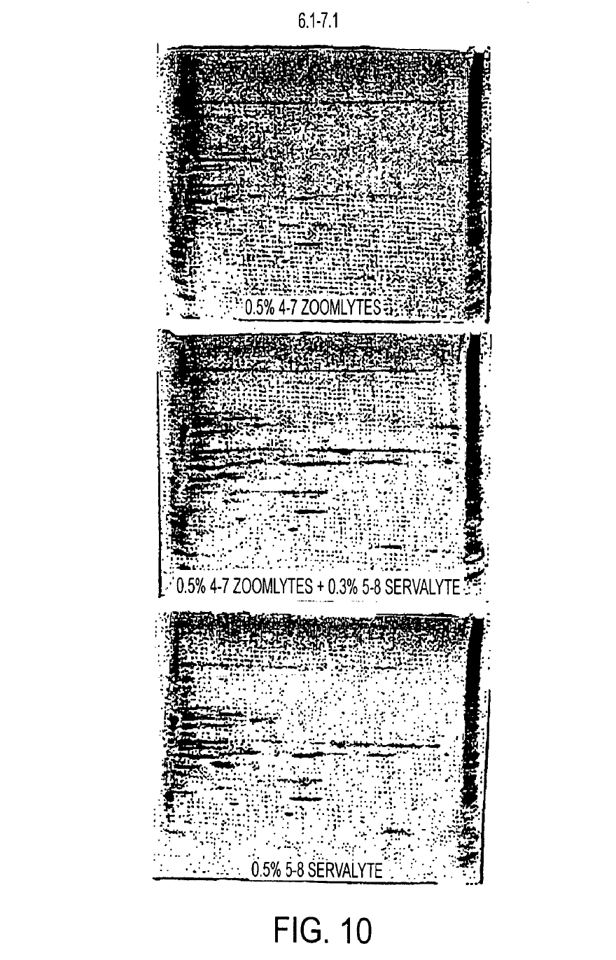

E. coli lysate was separated on 2D gels with narrow pH range strips in the first dimension for 30 μg, 100 μg and 300 μg loads using varied ampholyte types and concentrations. The 30 μg lysate separation with pH 4.5-5.5 strips showed good focusing with all ampholyte types used but with some acidic end streaking (FIG. 9). The 30 μg lysate separation with pH 5.3-6.3 strips showed better focusing in gels where narrow pH range (pH 5-7) ampholytes were incorporated in the rehydration solution (FIG. 9). The 30 μg lysate separation with pH 6.1-7.1 strips showed much better focusing in gels where pH 5-8 ampholytes were incorporated in the rehydration solutions (FIG. 10). The pH 6.1-7.1 gels were silver stained after Coomassie staining and destaining because of the low abundance of proteins in this pH range in E. coli lysate.

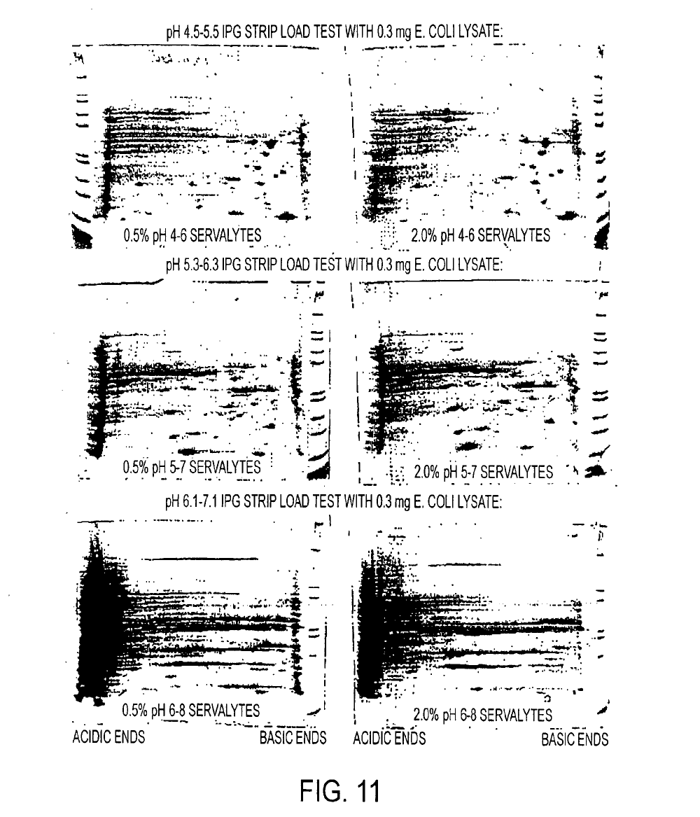

E. coli lysate separations using the higher 300 μg load showed good focusing despite some additional streaking (FIG. 11). The pH 4.5-5.5 gels performed well with all ampholyte concentrations (FIGS. 9 and 11) but the 5.3-6.3 gels showed reduced focusing with the 2.0% pH 5-7 ampholytes (FIG. 11). The 6.1-7.1 gels showed the best focusing with 0.5% pH 6-8 ampholytes (FIG. 11). Evaluation of the 6.1-7.1 gels was difficult, however, due to the low abundance of proteins in this pH range and the large acidic end pile-up when using the E. coli lysate sample. Resolution may have decreased due to an increase in conductive ions with 2% ampholytes, and longer run times may lead to better resolution. In some cases, this higher level of ampholytes is beneficial because it increases the solubility of some proteins.

FIG. 12 shows the type of overlap that can be expected for unfractionated protein lysates on 2D gels with the narrow range IPG strips. The top gel shows E. coli lysate separated on a 2D gel using a pH 4-7 IPG strip in the first dimension. The three lower 2D gels contained an identical sample separated in the first dimension on the three narrow pH range strips. A 100 μg total protein load was required for the 6.1-7.1 strip due to low level of protein in the lysate that pH region (shown in the 4-7 gel above). The expansion and overlap of the pH gradient is indicated by the vertical lines. Circles and arrows indicate the location of identical proteins on the pH 4-7 2D gel and narrow pH range 2D gels. Analysis of the protein spot pattern in the gels reveals a clear overlap between the pH 4.5-5.5 and pH 5.3-6.3 gels. The overlap between the pH 5.3-6.3 and pH 6.1-7.1 gels is difficult to visualize because of the heavy pile-up of the more acidic proteins in the lysate at the acidic end of the pH 6.1-7.1 gel. However, these results demonstrate that a very complex sample such as E. coli lysate may be directly applied to narrow pH range strips with good success.

### Example 3

### Rapid Rehydration of IPG Strips

### Materials and Methods

### Sample Preparation

E. coli cells were lysed by sonication in a solution containing 8 M deionized urea, 2% CHAPS, and 20 mM DTT. After centrifugation to remove insoluble debris, aliquots of the soluble fraction were stored at −80° C. Frozen aliquots were subsequently thawed and diluted as desired using the above urea, CHAPS and DTT concentrations. ZOOM® carrier ampholytes (Invitrogen, were added to achieve 0.5% v/v with the ampholyte pH range matching that of the IPG strip. Bromophenol blue was added as an indicator dye.

### Electrophoresis

Prepared samples (155 μl) were pipetted into ZOOM® IPGRunner™ cassettes followed by insertion of IPG strips. After rehydrating IPG strips for various times, isoelectric focusing was performed using a voltage ramp program set at 175V for 15 minutes, 175V to 2000V for 45 minutes and 2000V for either 30 minutes (pH 4-7 strips) or 105 minutes (pH 5.3-6.3 strips). IPG strips were then reduced using 50 mM DTT in 1×LDS Sample Buffer for 15 minutes and alkylated for 15 minutes using 125 mM iodoacetamide in 1×LDS Sample Buffer. The reduced and alkylated IPG strips were inserted into NuPAGE® ZOOM® IPG Gels and electrophoresed for 40-45 minutes at 200V before staining with SimplyBlue™ SafeStain.

### Spot Counting

E. coli proteins were visualized as spots on Coomassie-stained 2D gels. Corresponding square sections of imaged 2D gels were chosen for counting discrete spots (FIG. 14). Spot counting was performed with Phoretixm 2D Advanced Software, Version 5.1 (Nonlinear Dynamics Ltd.).

### In-Gel Digestion and Mass Spectrometry

Gel spots were excised and washed in 50% acetonitrile (“ACN”), 25 mM ammonium bicarbonate buffer until clear, then dehydrated in ACN and dried in a speedvac. Gel plugs were rehydrated with a minimal volume of trypsin solution (10 mg/mL in 25 mM ammonium bicarbonate buffer) and incubated overnight at 37° C. The digested peptides were extracted from the gel in two steps. The first extract was collected after incubating the gel pieces in 10 mL of 5% TFA for 30 minutes at room temperature. The second extract was collected after incubating the gel pieces in 10 mL of a 5% TFA/ACN solution for 30 minutes. The two extracts were pooled and dried in a Speedvac (Savant). The extracted and dried tryptic peptides were reconstituted in 50% ACN/0.1% TFA. Samples were then run on a VOYAGER-DE-STR MALDI-TOF instrument (ABI, Foster City, Calif.) using a-cyano-4-hydroxycinnamic acid (“CHCA”) as the matrix. Database searches were performed using ProFound (a software tool for searching a protein sequence database using information from mass spectra of peptide maps that is available at the Rockefeller University web site).

### Results

FIG. 14 shows 2D gel images for a time course of rehydration of IPG strips prior to isoelectric focusing and subsequent 2D gel electrophoresis. The actual sizes of the square sections used for spot counting were 3 cm×3 cm, and these sections comprise about ⅓ the area covered by the sample after 2D gel electrophoresis. Each pH 4-7 IPG strip was rehydrated with 75 μg of E. coli lysate in 155 μl of rehydration solution prior to isoelectric focusing. Each point in the rehydration time course was tested in duplicate, although only one of the replicates is shown. The four stained gels appear very similar in terms of focusing, protein migrations, spot intensities and numbers of spots.

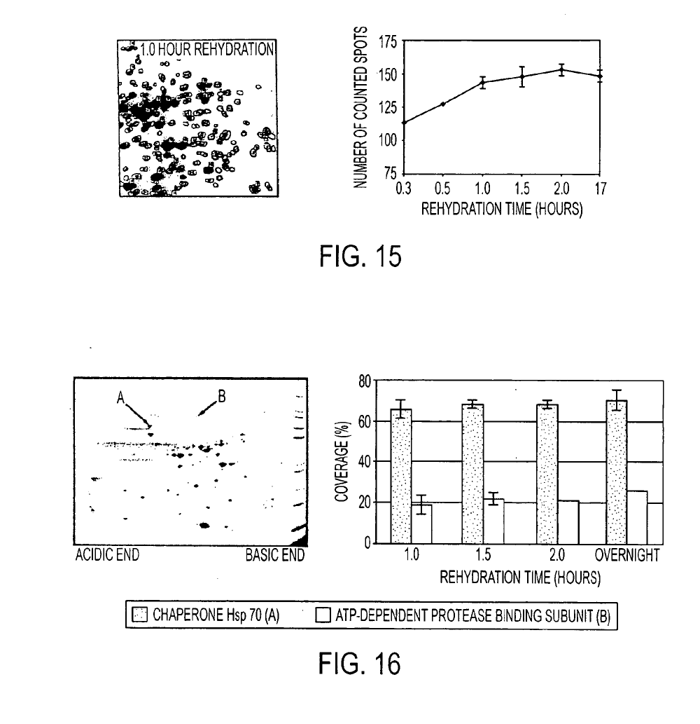

FIG. 15 consists of two parts. The left part shows an image exported from the Phoretix™ 2D software of a spot-counted 2D gel section. This particular comparison was between 2D gels that had been run using pH 4-7 IPG strips rehydrated either for 1 hour or overnight. The right part is a graphic representation of a rehydration time course charted in spreadsheet format by the number of counted spots. No significant reduction in the number of visualized proteins was observed for rehydration times as short as 1 hour.

FIG. 16 illustrates additional time-course results. Two visually correlated protein spots (A and B) from each 2D gel were excised for mass spectrometric analysis. No reduction in percent coverage was observed as a consequence of shortening the rehydration time.

FIG. 17 shows that short rehydration times can also be used for narrow pH range IPG strips. These experiments show that IPG strips of the current invention rehydrated for only 1 hour yield results equivalent to strips that are rehydrated overnight. The results further show that rapid rehydration does not negatively affect spot counting or mass spectrometry results. Rapid rehydration of IPG strips using the ZOOM® IPGRunner™ System thus enables fast, easy, and high-quality 2DE separations.

### Example 4

### Demonstration of Rapid Rehydration

Rehydration of a gel strip may be determined, for example, as follows. A volume of rehydration buffer containing rat liver lysate sample was added to the sample wells in an IPGRunner™ Cassette. Volumes from 110-170 uL were tested with the slots in an individual cassette containing the same volume of rehydration buffer. IPG strips of each pH range discussed herein, as shown in Table 2A and of known mass were inserted into the cassette to begin rehydration. IPG strips were removed individually after rehydration for time points of 0.5, 1.0, 1.5, 2.0, and 16 hours, blotted of excess rehydration solution and their mass measured. The gain in mass from the dehydrated strip to the rehydrated strip gave a measure of absorbed rehydration solution at each time point. Data were plotted, and duplicate strips were subjected to two-dimensional electrophoresis for visualization. All of the time points except 0.5 hours, yielded a mass gain that was at least 90% of the mass gain achieved at 16 hours.

This example provides evidence that gel strips of the present invention may rehydrate within 1 hour or less of contact with a rehydration solution.

All patents, patent publications, and other published references mentioned herein are hereby incorporated by reference in their entirety as if each had been individually and specifically incorporated by reference herein.

Examples are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such deviations and modifications that fall within the true spirit and scope of the invention.

1. A gel suitable for isoelectric focusing, comprising:
a polymerized acrylamide matrix cast from an acidic solution and a basic solution,
wherein the basic solution comprises at least three acrylamido buffers with a combined concentration of at least about 32 mM and the acrylamide matrix ranges in pH from not less than about pH 3.5 to not more than about pH 7.5.

2. The gel of claim 1, wherein the basic solution comprises at least three acrylamido buffers with a combined concentration of at least about 35 mM.

3. The gel of claim 1, wherein the basic solution comprises at least three acrylamido buffers with a combined concentration of at least about 40 mM.

4. The gel of any one of claims 1-3, wherein said gel has been dehydrated after casting.

5. The gel of claim 4, wherein said dehydrated gel is capable of rehydrating after contact with aqueous buffer in no more than 8 hours at room temperature.

6. The gel of claim 5, wherein said dehydrated gel is capable of rehydrating after contact with aqueous buffer in no more than 2 hours at room temperature.

7. The gel of claim 6, wherein said dehydrated gel is capable of rehydrating after contact with aqueous buffer in no more than 60 minutes at room temperature.

8. The gel of any one of claims 1-7, attached to a support.

9. The gel of claim 8, wherein said support is a plastic film.

10. The gel of claim 9, wherein said matrix and said support are fashioned as a strip.

11-24. (canceled)

25. A method for separating proteins of a sample using an electrophoretic field, comprising:
rehydrating a dried gel strip; and
isoelectrically focusing proteins of the sample within the rehydrated gel strip, wherein the method is performed in no more than 4 hours.

26. The method of claim 25, wherein the method is performed in about 3 hours.

27. The method of claim 25, further comprising placing the rehydrated gel comprising the isoelectrically focused proteins on a slab gel, and separating the proteins in a second dimension according to a characteristic other than isoelectric point, wherein the method is completed in no more than 8 hours.

28. The method of claim 25, wherein the method is completed in about 4 hours.

29. The method of claim 28, wherein proteins are separated in the second dimension based on their molecular weight.

30. The method of claim 25, wherein the rehydrated gel strip has a buffer capacity beta value of at least 3 mEq/L/pH.

31. The method of claim 30, wherein the rehydrated gel strip has a buffer capacity beta value of between 4 and 6 mEq/L/pH.

32. A gel having a pH that varies progressively along the length of said gel, comprising:
a polymer matrix cast from an acidic solution and a basic solution, said polymer matrix comprising at least one polyacrylamide species and said gel, once dried, is capable of rehydrating in no more than about 8 hours at room temperature.

33. The gel of claim 32, wherein said gel, once dried, is capable of rehydrating in not more than about 2 hours at room temperature.

34. The gel of claim 33, wherein said gel, once dried, is capable of rehydrating in not more than about 60 minutes at room temperature.

35. The gel of claim 32, wherein said gel has a buffering capacity beta value of greater than 3 mEq/L/pH.

36. The gel of claim 35, wherein the buffering capacity beta value is 5 mEq/L/pH.

37. The gel of claim 35, wherein said gel has a pH that ranges from not less than about pH 3.5 to not more than about pH 7.5.

38-126. (canceled)
